(19) United States
(12) Patent Application Publication
(10) Pub. No.: US 2024/0174645 A1
(43) Pub. Date: May 30, 2024

(54) COMPOUND, PRODUCTION METHOD THEREFOR, COMPLEX, AND SHORT WAVELENGTH INFRARED FLUORESCENT AGENT

(71) Applicant: RIKEN, Saitama (JP)

(72) Inventors: Takashi Jin, Saitama (JP); Manhadeva Swamy MAKANAHALLI MADEGOWDA, Saitama (JP); Kenji MONDE, Hokkaido (JP); Yuta MURAI, Hokkaido (JP)

(21) Appl. No.: 18/282,174

(22) PCT Filed: Mar. 15, 2022
(86) PCT No.: PCT/JP2022/011674

§ 371 Date: Sep. 14, 2023

(30) Foreign Application Priority Data
Mar. 15, 2021 (JP) 2021-041717

(51) Int. Cl. C07D 403/06; A61K 49/00; C07D 403/14

(52) U.S. Cl. CPC C07D 403/06; A61K 49/0032; A61K 49/0058; C07D 403/14

(57) ABSTRACT

A novel compound that enables shortwave infrared bioimaging is provided. A compound in accordance with an aspect of the present invention is represented by a formula (1) below. In the formula (1), n represents an integer of 3 to 5, X represents a salt of a sulfonic acid group or a reactive crosslinking group with respect to a molecular recognition agent.

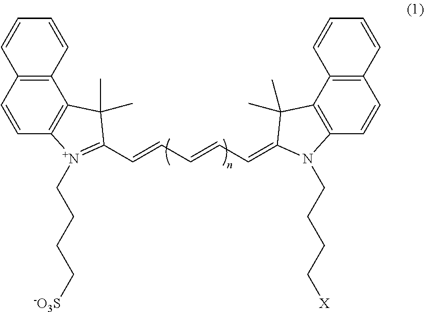

### TECHNICAL FIELD

The present invention relates to a compound, a method for producing the compound, a complex, and a shortwave infrared fluorescent agent.

### BACKGROUND ART

As a bioimaging technique for non-invasively visualizing a living body, a method for visualizing a blood vessel, a tumor, or the like with use of a fluorescent agent which emits near-infrared light having a wavelength of 700 nm to 900 nm has been widely used. As an active component of a fluorescent agent which can be applied to bioimaging, indocyanine green (ICG), which emits near-infrared fluorescence, is known.

Meanwhile, near-infrared light is easily scattered and can easily be absorbed into tissues. Therefore, in bioimaging using a fluorescent agent that emits near-infrared light, it is difficult to clearly visualize a microstructure located deeper, by 1 cm or more, for example, from the surface of a target living body or a target biological tissue. Therefore, a fluorescent agent which emits shortwave infrared light having a wavelength of 900 nm to 1400 nm, which is less likely to scatter, is demanded. In this regard, the fluorescence of ICG is known to extend to a wavelength region of shortwave infrared light (see, for example, Non-Patent Literature 1).

### CITATION LIST

### Non-Patent Literature

[Non-Patent Literature 1]

Setsuko Tsuboi and Takashi Jin, RSC Advances, p. 28171-28179, 2020

### SUMMARY OF INVENTION

### Technical Problem

However, the fluorescence intensity of ICG in the shortwave infrared region is small. Therefore, in shortwave infrared bioimaging using a fluorescent agent containing ICG, it may be difficult to clearly visualize a microstructure located deeper, by 1 cm or more, from the surface of a living body. Therefore, a fluorescent agent which can be applied to shortwave infrared bioimaging technique is demanded.

An object of an aspect of the present invention is to provide a novel technique that makes it possible to perform shortwave infrared bioimaging.

### Solution to Problem

In order to attain the object, a compound in accordance with an aspect of the present invention is represented by the following formula (1). In the formula (1), n represents an integer of 3 to 5, X represents a salt of a sulfonic acid group or a reactive crosslinking group with respect to a molecular recognition agent.

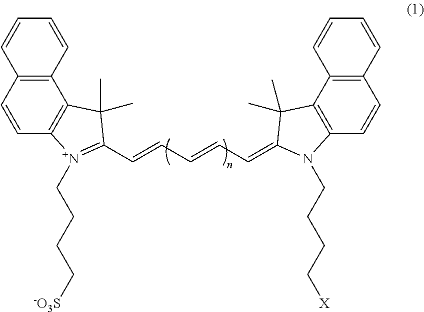

In order to attain the object, in a complex in accordance with an aspect of the present invention, the compound in accordance with an aspect of the present invention is bonded to a molecular recognition agent via a residue of the reactive crosslinking group in the compound.

In order to attain the object, a shortwave infrared fluorescent agent in accordance with an aspect of the present invention contains the compound in accordance with an aspect of the present invention and/or the complex in accordance with an aspect of the present invention.

In order to attain the object, a method in accordance with an aspect of the present invention for producing a compound represented by the following formula (1) includes a first step of synthesizing a first compound represented by the following formula (1a), a second step of replacing an anilino group in the formula (1a) with a structure represented by the following formula (1b), and a third step of replacing a phenylimino group in the formula (1a) with a structure represented by the following formula (1c). In the following formula, n represents an integer of 3 to 5, X represents a salt of a sulfonic acid group or a reactive crosslinking group with respect to a molecular recognition agent.

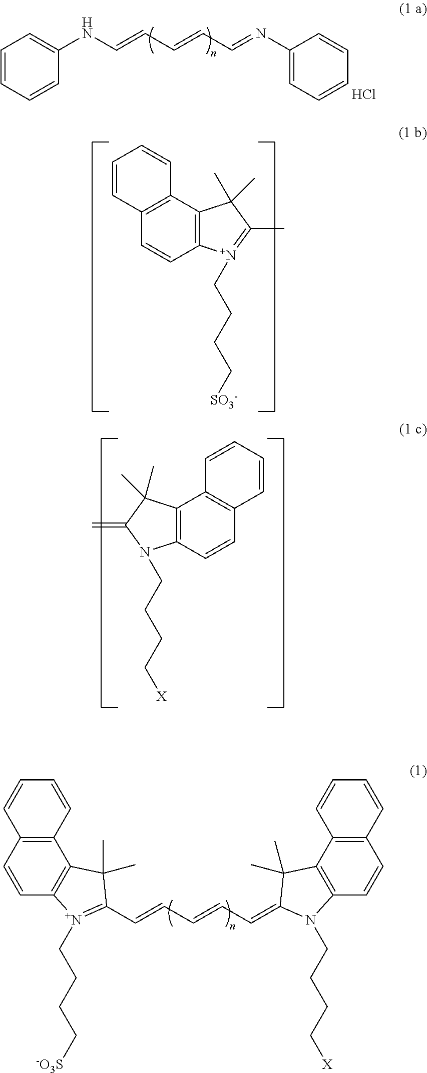

### Advantageous Effects of Invention

With an aspect of the present invention, it is possible to provide a novel technique that enables shortwave infrared bioimaging.

### BRIEF DESCRIPTION OF DRAWINGS

FIG. 1 is a view showing a relationship between the concentration of a compound 4 in Examples in the present invention and cell viability.

FIG. 2 is a view showing a relationship between the concentration of a compound 10 in Examples in the present invention and cell viability.

FIG. 3 is a view showing photographs that represent bright-field images and shortwave infrared fluorescence images of the lower limb and abdomen of a hairless mouse in which a shortwave infrared fluorescent agent 1 was used as an optical contrast medium in Examples in the present invention.

FIG. 4 is a view showing photographs that represent bright-field images and shortwave infrared fluorescence images of the lower limb and abdomen of a hairless mouse in which a shortwave infrared fluorescent agent 2 was used as an optical contrast medium in Examples in the present invention.

FIG. 5 is a view showing photographs that represent bright-field images and shortwave infrared fluorescence images of the head top parts of hairless mice in which the shortwave infrared fluorescent agent 1 and the shortwave infrared fluorescent agent 2 in Examples in the present invention and a shortwave infrared fluorescent agent C1 in a comparative example of the present invention were used as optical contrast media.

FIG. 6 is a view showing photographs that represent bright-field images and shortwave infrared fluorescence images of the lower limb and abdomen of a hairless mouse in which a shortwave infrared fluorescent agent 3 was used as an optical contrast medium in Examples in the present invention.

FIG. 7 is a view showing photographs that represent bright-field images and shortwave infrared fluorescence images of the lower limb and abdomen of a hairless mouse in which a shortwave infrared fluorescent agent 4 was used as an optical contrast medium in Examples of the present invention.

FIG. 8 is a view showing photographs that represent bright-field images and shortwave infrared fluorescence images of the head top parts of hairless mice in which the shortwave infrared fluorescent agent 3 and the shortwave infrared fluorescent agent 4 in Examples in the present invention and a shortwave infrared fluorescent agent C2 in a comparative example of the present invention were used as optical contrast media.

FIG. 9 is a view showing the fluorescence spectra of a complex 1 and a complex 2 in Examples in the present invention.

FIG. 10 is a view showing photographs that represent a bright-field image and a shortwave infrared fluorescence image of the tumor part of a cancer-bearing mouse in which a shortwave infrared fluorescent agent 5 was used as an optical contrast medium in Examples in the present invention.

FIG. 11 is a view showing photographs that represent bright-field images and shortwave infrared fluorescence images of the cancer tumor, heart, kidney, spleen, and liver of a cancer-bearing mouse in which the shortwave infrared fluorescent agent 5 was used as an optical contrast medium in Examples in the present invention.

FIG. 12 is a view showing photographs that represent a bright-field image and a shortwave infrared fluorescence image of the tumor part of a cancer-bearing mouse in which a shortwave infrared fluorescent agent 6 was used as an optical contrast medium in Examples in the present invention.

FIG. 13 is a view showing photographs that represent bright-field images and shortwave infrared fluorescence images of the cancer tumor, heart, kidney, spleen, and liver of a cancer-bearing mouse in which the shortwave infrared fluorescent agent 6 was used as an optical contrast medium in Examples in the present invention.

FIG. 14 is a view showing photographs that represent a bright-field image and a shortwave infrared fluorescence image of the tumor part of a cancer-bearing mouse in which a shortwave infrared fluorescent agent 7 was used as an optical contrast medium in Examples in the present invention.

FIG. 15 is a view showing photographs that represent bright-field images and shortwave infrared fluorescence images of the cancer tumor, heart, kidney, spleen, and liver of a cancer-bearing mouse in which the shortwave infrared fluorescent agent 7 was used as an optical contrast medium in Examples in the present invention.

FIG. 16 is a view showing photographs that represent bright-field images and shortwave infrared fluorescence images of the tumor parts of cancer-bearing mice in which a shortwave infrared fluorescent agent 8 and a shortwave infrared fluorescent agent 9 were used as optical contrast media in Examples in the present invention.

FIG. 17 is a view showing photographs that represent bright-field images and shortwave infrared fluorescence images of the tumor parts of cancer-bearing mice in which a shortwave infrared fluorescent agent 10 and a shortwave infrared fluorescent agent 11 were used as optical contrast media in Examples in the present invention.

FIG. 18 is a view showing photographs that represent bright-field images and shortwave infrared fluorescence images of the tumor parts of a cancer-bearing mouse into which Kadcyla was injected and a cancer-bearing mouse into which Kadcyla was not injected, captured by using a shortwave infrared fluorescent agent 12 and a shortwave infrared fluorescent agent 13 for optical contrast media, in Examples in the present invention.

FIG. 19 is a view showing photographs that were captured after specific periods of time elapsed and that represent bright-field images and shortwave infrared fluorescence images of the tumor part of a cancer-bearing mouse in which a shortwave infrared fluorescent agent 13 was used as an optical contrast medium and into which Kadcyla was injected in Examples in the present invention.

### DESCRIPTION OF EMBODIMENTS

The following description will discuss an embodiment of the present invention in detail.

### Compound

A compound in accordance with an embodiment of the present invention is represented by the following formula (1):

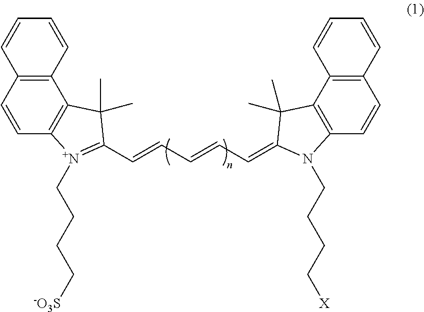

where n represents an integer of 3 to 5. n can be determined as appropriate from the viewpoint of allowing the compound of the formula (1) to emit shortwave infrared fluorescence and to exhibit sufficient water-solubility. From the above viewpoint, n is preferably 3 or 4.

In an embodiment of the present invention, the term “shortwave infrared” means electromagnetic waves having a wavelength of 900 nm to 1400 nm. The compound of the formula (1), according to the structure thereof (e.g., n), has the peak of fluorescence intensity in a wavelength region of shortwave infrared. Therefore, the wavelength in detecting the fluorescence of the compound of the formula (1) may be determined as appropriate within a range where the compound of the formula (1) emits fluorescence that has sufficiently high shortwave infrared intensity. In addition, the wavelength of excitation light in the fluorescence of the compound of the formula (1) need only be the wavelength of light by which the compound of the formula (1) is excited. The excitation wavelength can be determined as appropriate within a range of, for example, 900 nm to 1100 nm according to the structure (e.g., n) of the compound of the formula (1), the depth of the detection target site, or the like.

In the formula (1), X represents a salt of a sulfonic acid group or a reactive crosslinking group with respect to a molecular recognition agent. The salt of the sulfonic acid group is not limited, and can be, for example, an alkali metal salt, and more specifically, can be a sodium salt.

The reactive crosslinking group is a functional group to be bonded to a molecular recognition agent. The bonding to the molecular recognition agent need only be a bond which is appropriate according to the application of the compound of the formula (1). For example, the bonding can be a covalent bond or a hydrogen bond. The bonding of the reactive crosslinking group to the molecular recognition agent can be formed by the molecular recognition agent bonding to the reactive crosslinking group, or can be formed by a part of the reactive crosslinking group bonding to the molecular recognition agent while involving the desorption of a part of the reactive crosslinking group.

The reactive crosslinking group is preferably a functional group to be crosslinked with the molecular recognition agent in a mild conditions, such as mixing at room temperature, from the viewpoint of stability and easy handleability. From such viewpoints, the reactive crosslinking group is preferably at least one organic group selected from the group consisting of an N-hydroxysuccinimide ester group, a maleimide group, an alkynyl group, and an azide group.

The alkynyl group preferably has not more than 5 carbon atoms, from the viewpoint of, for example, water-solubility of the compound of the formula (1). However, the alkynyl group is not limited to this feature. Examples of the alkynyl group include an ethynyl group and a propynyl group.

The ester structure of the N-hydroxysuccinimide ester group need only be a structure which can be bonded to a linear-chain hydrocarbon group or which is derivable from an organic group containing a linear-chain hydrocarbon group. For example, the ester structure of the N-hydroxysuccinimide ester group can be an N-hydroxysuccinimide carbonyl group. In addition to the above organic group, the reactive crosslinking group can further have another molecular structure which can constitute the compound of the formula (1), provided that the effects of the present embodiment can be obtained. The reactive crosslinking groups other than the N-hydroxysuccinimide ester group can also further have a structure (e.g., an alkyl group, an amide group, and the like) which can be bonded to a linear-chain hydrocarbon group or which is derivable from an organic group containing a linear-chain hydrocarbon group.

The above reactive crosslinking group can be easily bonded to a molecular recognition agent having an amino group, a sulfhydryl group, an azide group, or an alkynyl group. Therefore, the reactive crosslinking group is preferable from the viewpoint of easy and accurate introduction, into the compound of the formula (1), a part for identifying a biological tissue.

Alternatively, the reactive crosslinking group can be introduced into the compound of the formula (1) by bonding of a linker material for use in the bonding of a fluorescent substance to amino acid. In such a case, a reactive crosslinking group which can be bonded to an amino group of the molecular recognition agent can be introduced into the compound of the formula (1). Examples of the linker material include 6-aminocaproic acid, 2-aminoadipic acid, 3-aminoadipic acid, 4-aminobutyric acid, 5-aminovaleric acid, 7-aminoheptanoic acid, 8-aminooctanoic acid, 11-aminoundecanoic acid, 12-aminododecanoic acid, 2-aminobenzoic acid, 3-aminobenzoic acid, and 4-aminobenzoic acid.

The molecular recognition agent is a component having a molecular structure capable of identifying a specific molecule. The molecular recognition agent need only be a component containing a molecular structure which can be bonded to a specific molecule, and one type of the molecular recognition agent or two or more types of molecular recognition agents can be used. The molecular recognition agent has a part to be bonded to a reactive crosslinking group. Examples of the part include an amino group, a sulfhydryl group, an azide group, and an alkynyl group. Examples of the molecular recognition agent include a peptide, a protein, a nucleic acid derivative, an antibody, a fragment of with antibody with an antigen-binding capacity, and a cell.

The compound of the formula (1) such as the one described above, more specifically, can be represented by one of the following formulas (2) to (9):

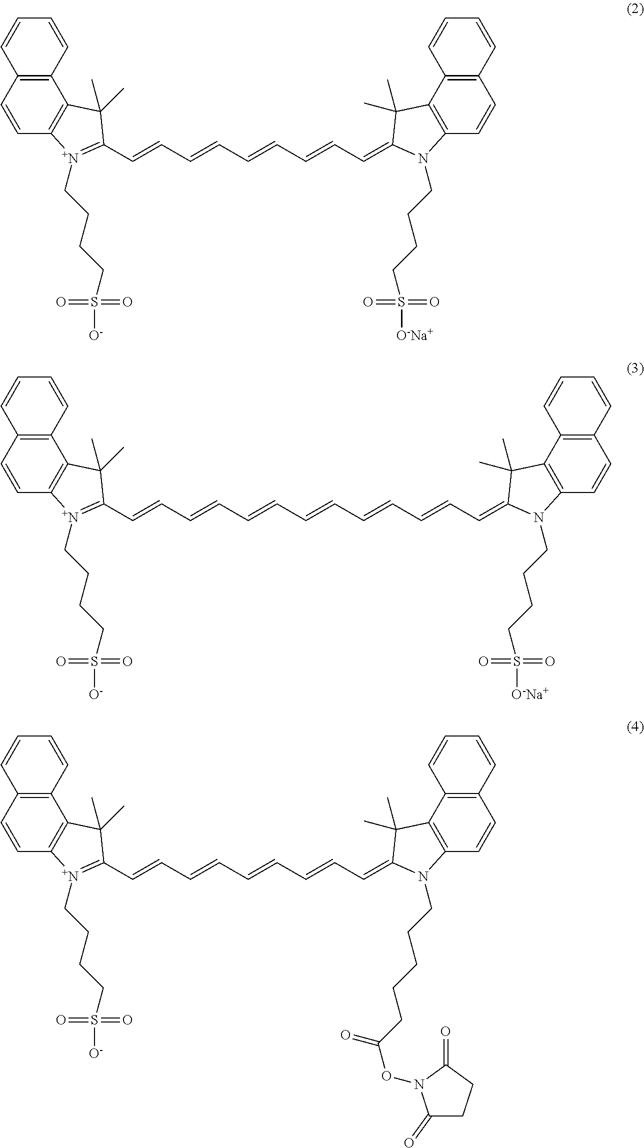

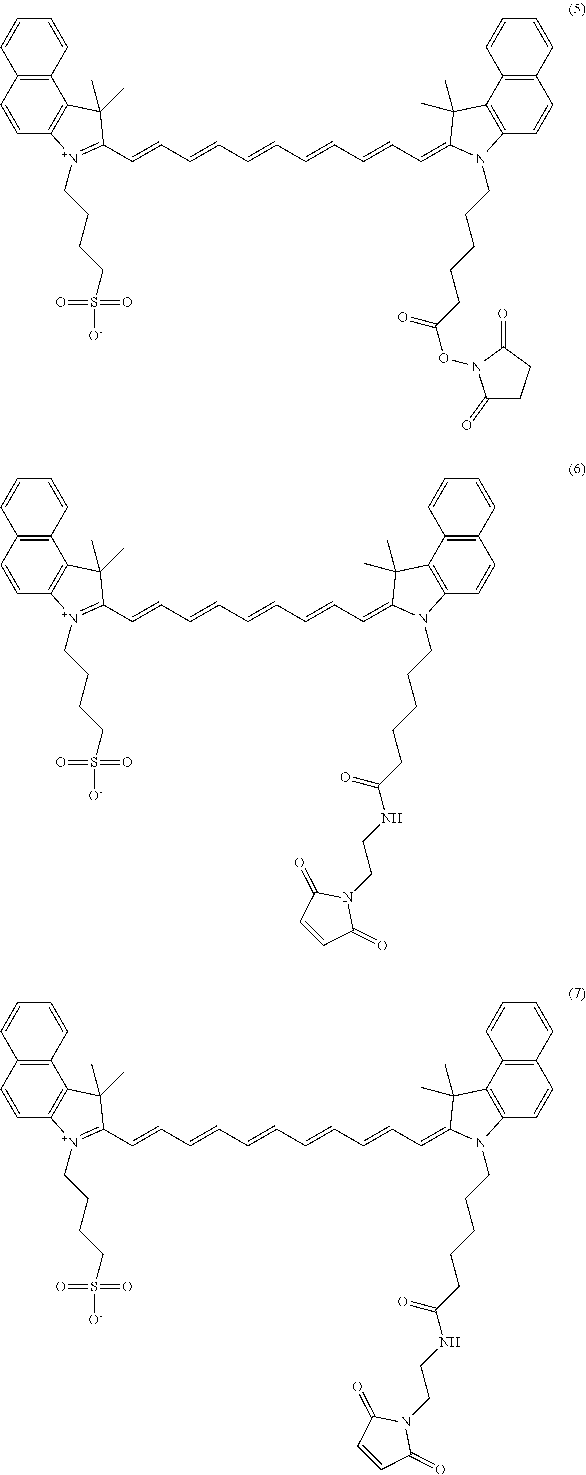 [SMILES: CC1(C)C(/C=C/C=C/C=C/C=C/C=C/C=C2/N(CCCCCC(=O)ON3C(=O)CCC3=O)c3ccc4ccccc4c3C2(C)C)=[N+](CCCCS(=O)(=O)[O-])c2ccc3ccccc3c21 ; CC1(C)C(/C=C/C=C/C=C/C=C/C=C2/N(CCCCCC(=O)NCCN3C(=O)C=CC3=O)c3ccc4ccccc4c3C2(C)C)=[N+](CCCCS(=O)(=O)[O-])c2ccc3ccccc3c21 ; CC1(C)C(/C=C/C=C/C=C/C=C/C=C/C=C2/N(CCCCCC(=O)NCCN3C(=O)C=CC3=O)c3ccc4ccccc4c3C2(C)C)=[N+](CCCCS(=O)(=O)[O-])c2ccc3ccccc3c21]

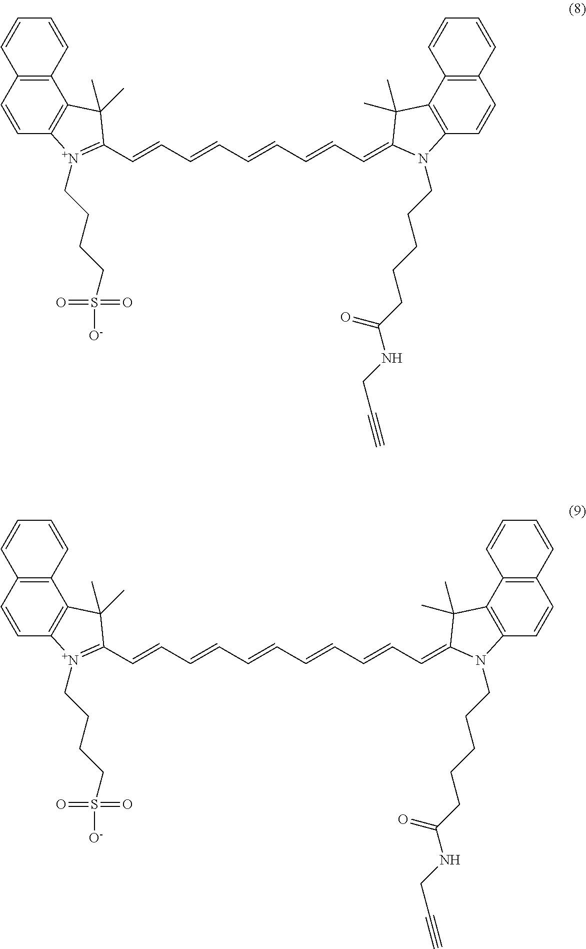 [SMILES: C#CCNC(=O)CCCCCN1/C(=C/C=C/C=C/C=C/C=C/C2=[N+](CCCCS(=O)(=O)[O-])c3ccc4ccccc4c3C2(C)C)C(C)(C)c2c1ccc1ccccc21 ; C#CCNC(=O)CCCCCN1/C(=C/C=C/C=C/C=C/C=C/C=C/C2=[N+](CCCCS(=O)(=O)[O-])c3ccc4ccccc4c3C2(C)C)C(C)(C)c2c1ccc1ccccc21]

The compound of the formula (1) is an organic dye that emits shortwave infrared fluorescence. Such an organic dye is expected to be applied to bioimaging as described above. Therefore, research has been conducted on, as shortwave infrared fluorescent agents, compounds having various molecular frameworks (e.g., Jin T, ECS J. Solid State Sci. Technol, 8, R9-R13 (2019)). Thus, conventionally, a compound having shortwave infrared fluorescence as a fluorescence characteristic specific to a molecular framework has been researched.

Meanwhile, in a fluorescent compound, a part of energy absorbed from excitation light is generally converted into vibrational energy of a molecule, and then consumed. Then, it is considered that when the molecular structure becomes larger, the above absorbed energy is converted and the greater proportion of the energy is consumed. According to such a general tendency, it is expected that, when the chain-like structure of ICG is lengthened, fluorescence emission efficiency is reduced more.

However, the compound of the formula (1) has a structure similar to that of ICG and has a chain-like structure longer than that of ICG, while having, in shortwave infrared fluorescence, light emission efficiency equivalent to or higher than that of fluorescence in near-infrared of ICG. Therefore, the compound of the formula (1) is a unique compound that, regardless of having a molecular structure larger than that of ICG, does not apply to the general tendency described above.

### Method for Producing Compound

The compound of the formula (1) can be produced by a production method including first to third steps below. In the description of the production method below, the meanings of the symbols in the general formula are identical to those in the aforementioned compounds.

The first step is a step of synthesizing a first compound represented by the following formula (1a): As described above, n represents an integer of 3 to 5.

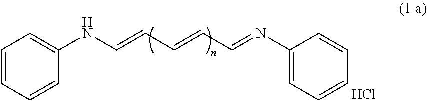

The compound of the formula (1a) can be synthesized by reacting a linear-chain diene aldehyde in the presence of a phosphorous compound so as to elongate the linear-chain diene structure as appropriate, and then reacting a resultant product with aniline. The synthesis can be efficiently performed by adjusting the temperature as appropriate. In the elongation of the linear-chain diene structure, from the viewpoint of, for example, the reactivity, solubility, or stability of the intermediate product, the terminal aldehyde of the linear-chain diene structure in the intermediate product may be temporarily substituted with another structure, such as a carboxylic acid ester or an alcohol.

The second step is a step of replacing an anilino group in the formula (1a) with a structure represented by the following formula (1b):

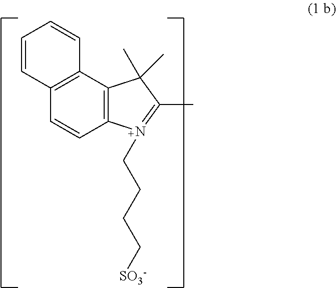

The anilino group in the formula (1a) can be replaced with the structure of the formula (1b) by reacting, in the presence of acetic anhydride and sodium acetate, the compound represented by the following formula (1b1) with a compound of the formula (1a) or a compound in which the phenylimino group in the formula (1a) is replaced with a structure represented by a formula (1c) below. The compound of the formula (1b1) is obtained by reacting 1,1,2-trimethyl-1H-benzo[e]indole with 1,4-butanesultone.

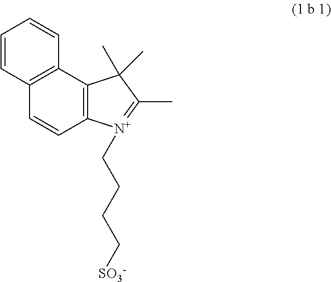

The third step is a step of replacing the phenylimino group in the formula (1a) with the structure represented by the following formula (1c):

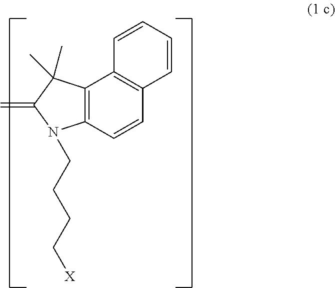

In a case where X is a salt of a sulfonic acid group, the phenylimino group in the formula (1a) can be replaced with the structure of the formula (1c) in a manner similar to the replacement of the anilino group in the above formula (1a), using, as a raw material, the compound of the formula (1a) or a compound in which the phenylimino group in the formula (1a) is replaced by the structure represented by the formula (1b).

In a case where X is the reactive crosslinking group described above, the phenylimino group in the formula (1a) can be replaced with the structure represented by the formula (1c) by first replacing the phenylimino group with a structure represented a formula (1c1) below and then bonding the reactive crosslinking group to the terminal carboxyl group or replacing the carboxyl group with the reactive crosslinking group.

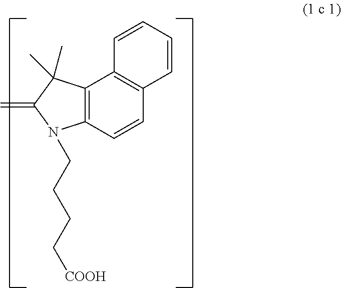

The phenylimino group in the formula (1a) can be replaced with the structure represented by the formula (1c1) in a manner similar to the above-described replacement of the anilino group in the formula (1a) by, for example, using a bromine salt of a compound in which the sulfonic acid group in the formula (1b1) is replaced with the carboxyl group. The reaction in which the reactive crosslinking group is introduced into the carboxyl group after the replacement to the structure represented by the formula (1c1) can be performed by the application of a known method according to the target reactive crosslinking group.

In the above production method, the order in which the second step and the third step are to be carried out can be determined as appropriate from the viewpoint of, for example, the solubility of a raw material or a product or the reactivity of the raw material, provided that the target compound of the formula (1) can be produced.

For example, in a case where the target compound is the compound of the formula (2), that is, in a case where n is 3 and X is a salt of a sulfonic acid group in the above formula, the second step and the third step can be carried out simultaneously. The second step and the third step described above can be carried out simultaneously by, for example, reacting two or more equivalents of the compound of the formula (1b1) with respect to the compound of the formula (1a) under the conditions of the second step. Carrying out the second step and the third step simultaneously makes it possible to more easily produce the compound of the formula (1), and is also advantageous from the viewpoint of suppressing a decrease in yield due to an increase in the number of steps.

For example, in a case where the target compound is the compound of the formula (3), that is, in a case where n is 4 and X is a salt of a sulfonic acid group in the above formula, it is preferable to carry out the third step after the second step. In addition, in a case where the target compound is the compound of the formula (4), that is, in a case where n is 3 and X is a reactive crosslinking group in the above formula, it is preferable to carry out the third step after the second step. In these cases, the third step need only be carried out using, as a raw material, the product of the second step. Carrying out the third step after the second step in the production of the compound of the formula (3) or the compound of the formula (4) is suitable from the viewpoint of increasing the yield of the replacement of the phenylimino group in the formula (1a) in the third step.

In a case where the target compound is the compound of the formula (5), that is, in a case where n is 4 and X is a reactive crosslinking group in the above formula, it is preferable to carry out the third step before the second step. In this case, the second step need only be carried out using, as a raw material, the product of the third step. Carrying out the third step before the second step in the production of the compound of the formula (5) is suitable from the viewpoint of increasing the yield in the replacements of the anilino group and the phenylimino group in the formula (1a).

The above production method can further include a step other than the first step through the third step described above, provided that the effects of the present embodiment can be obtained. For example, the above production method can further include a step of purifying a product in each step. Such a purification step can be carried out as appropriate by a known method such as washing with a solvent or column chromatography.

In the above production method, the compound of each formula can be synthesized using a known technique. However, it is preferable to select, as appropriate, synthesis conditions from the viewpoint of reactivity and productivity.

For example, the synthesis reaction of the compound of the formula (1a), the second step, and the third step are promoted at a high temperature. Meanwhile, in a case where the reaction temperature is increased, a target compound having a chain-like structure of a desired length and a by-product having a chain-like structure shorter than that of the target compound may be generated at substantially equal proportions.

The target compound and the by-product have similar physical properties such as polarity. It can easily be expected that it is difficult to separate the target compound from the mixtures of the target compound and the by-product. In fact, physical properties are similar, and it is difficult to isolate the target compound from the mixture.

In the synthesis reaction of the compound of the formula (1a), the second step, and the third step, reducing the reaction temperature also reduces the reaction speed, but makes it possible to synthesize the target compound at a higher proportion. For example, as demonstrated in Examples described later, in a case where a reaction in which a chain-like structure is lengthened is made intentionally at room temperature, it is possible to efficiently obtain a target compound with high purity, although the reaction speed is not high. Thus, in the above production method, it is possible to highly efficiently obtain a target compound by, while utilizing a known technique, properly adjusting the synthesis conditions, such as a reaction temperature, as necessary, from the viewpoint of reactivity and productivity.

The above synthesis reaction in an embodiment of the present invention will be described in more detail, using specific examples. In the synthesis of ICG which has been conventionally used for bioimaging, ordinarily, the anilino group of the compound of the formula (1a) in which n is 2 is replaced with a sulfonic acid compound represented by the formula (1b), and then the structure of the formula (1c) is introduced into the sulfonic acid compound. In a case the method for producing the compound in accordance with the present embodiment is applied to the synthesis of ICG, the second step, the third step described above will be carried out in this order. In the synthesis of ICG, the solubility of a product markedly deteriorates as the synthesis proceeds, particularly, in a case where the substitution with the sulfonic acid compound is performed. Therefore, in the ordinary method for synthesizing ICG, a synthesis reaction in which the compound of the formula (1b) and the structure of the formula (1c) are introduced is made at a higher temperature.

Meanwhile, in an embodiment of the present invention, the carbon chain (linear-chain polyene chain) in the compound of the formula (1a) to be used is longer in comparison with the case of the synthesis of ICG. In particular, in the compound of the formula (1a) which is used in synthesizing the compound (ICG-C11-NHS)of the formula (5), n is 4. This indicates an even longer linear-chain polyene chain. Therefore, in the production method in accordance with the present embodiment, the compound of the formula (1a) is clearly lower in stability in comparison with the compound of the formula (1a) in the synthesis of ICG. Therefore, in synthesizing the compound of the formula (1c) after the introduction of the compound of the formula (1b), performing these syntheses at a high temperature leads to the production, at a high proportion, of a by-product in which the number of carbon atoms of the polyene chain is lower by 2 than that of the target compound (approximate ratio between the target compound and the by-product=1:1). Because the target compound and the by-product are extremely close in polarity, it is not preferable to obtain a target compound by the separation of these compounds, from the viewpoint of yield.

Therefore, as described above, in the synthesis of particularly the compound of the formula (5), it is preferable to carry out the third step before the second step. In such synthesis, the third step which is carried out first leads to the production of a product which is more soluble in a solvent. This makes it possible to carry out the subsequent second step at room temperature or at a lower temperature than room temperature. As a result, the production of the by-product is suppressed in the second step, and it is possible to substantially obtain only the target compound. A synthesis method in which the steps are carried out in such an order is, although the reaction ratio of the target compound is slightly low, preferable from the viewpoint of obtaining the target compound with high purity.

### Complex

In the complex in accordance with an embodiment of the present invention, the above-described compound in accordance with the present embodiment is bonded to a molecular recognition agent via a residue of the reactive crosslinking group in the compound. The meanings of the reactive crosslinking group and the molecular recognition agent are as described above in the description of the compound in accordance with an embodiment of the present invention. From the viewpoint of the application to molecular imaging, the molecular recognition agent is preferably a component which can be bonded to a specific part of a living body. From such a viewpoint, the molecular recognition agent is preferably an antibody or a fragment of the antibody with an antigen-binding capacity, among those exemplified above. A complex which includes bonding to such an antibody or a fragment is useful as a fluorescence marker for a specific biological tissue such as a tumor. In addition, the complex can be used to detect a tumor and confirm a pharmacological effect or a therapeutic effect on a tumor by molecular imaging with shortwave infrared.

### Shortwave Infrared Fluorescent Agent

A shortwave infrared fluorescent agent in accordance with an embodiment of the present invention is a composition that contains the above-described compound of the formula (1) and/or the above-described complex. One type of the compound of the formula (1) in the shortwave infrared fluorescent agent in accordance with an embodiment of the present invention or two or more types of the compounds can be used. Also one type of the complex or two or more types of the complexes can be used. In the shortwave infrared fluorescent agent in accordance with the present embodiment, the compound of the formula (1) or the complex containing the compound has a property of emitting shortwave infrared. Therefore, the shortwave infrared fluorescent agent in accordance with an embodiment of the present invention can be used for shortwave infrared fluorescence imaging.

The shortwave infrared fluorescent agent in accordance with the present embodiment can further contain a component other than the above-described compound of the above formula (1) and the complex, provided that the effects of the present embodiment can be obtained. For example, the shortwave infrared fluorescent agent can be used as a solution of phosphate buffered saline, because the shortwave infrared fluorescent agent is ordinarily used in a living body.

It is also known that the stability of ICG and the complex thereof in aqueous solution is improved in the presence of bovine serum albumin (BSA), and that the emission intensity of fluorescence of ICG and the complex thereof is increased in the presence of BSA (see, for example, “Takashi Jin et al., Med. Chem. Commun, 2016, 7, 623-631”). The same is true with the shortwave infrared fluorescent agent in an embodiment of the present invention. That is, the shortwave infrared fluorescent agent in an embodiment of the present invention preferably contains BSA, from the viewpoint of increasing the stability of the compound and the complex in accordance with the present embodiment in aqueous solution and from the viewpoint of increasing the emission intensity of shortwave infrared fluorescence.

The concentration of the compound or the complex in accordance with the present embodiment in the shortwave infrared fluorescent agent need only be a concentration at which sufficiently emission intensity of fluorescence is achieved according to the application of the shortwave infrared fluorescent agent. For example, in the application for bioimaging or molecular imaging, the concentration of the compound or the complex in accordance with the present embodiment in the shortwave infrared fluorescent agent is preferably 0.1 μM or more, more preferably 1 μM or more, and even more preferably 10 μM or more, from the viewpoint of forming a sufficiently distinct image in bioimaging or molecular imaging with shortwave infrared. In the application for bioimaging or molecular imaging, the concentration of the compound or the complex in accordance with the present embodiment in the sub-shortwave infrared fluorescent agent can be 100 μM or less, 10 μM or less, or 1 μM or less, from the above viewpoint.

The BSA content of the shortwave infrared fluorescent agent can be determined as appropriate, provided that the effect of increasing the fluorescence emission intensity can be obtained. From such a viewpoint, the BSA content can be 1 mg/mL to 100 mg/mL.

In addition to BSA, a component which forms a micelle with the shortwave infrared fluorescent agent can be expected to bring about the above effect as in the case of the use in combination with BSA. From such a viewpoint, examples of similar components other than BSA include phospholipids such as human serum albumin (HSA) and lecithin, and long-chain fatty acids.

### Applications

The shortwave infrared fluorescent agent in an embodiment of the present invention functions as an excellent optical contrast medium in fluorescence imaging of a living body with shortwave infrared light having a wavelength region of 900 nm or more. In fluorescence imaging of a living body, it is possible to determine, as appropriate, the wavelength of excitation light and the fluorescence detection wavelength, within the above-described ranges in the description of the compound. In a case where a plurality of different combinations of excitation wavelengths and fluorescence detection wavelengths are set, it is possible to perform imaging of a plurality of biological tissues or biomolecules with different depths, by a single administration of the shortwave infrared fluorescent agent into a living body.

The shortwave infrared fluorescent agent containing the complex in an embodiment of the present invention can be easily prepared and can be used as an optical contrast medium for molecular imaging.

By using the shortwave infrared fluorescent agent in an embodiment of the present invention as an optical contrast medium, it is possible to use shortwave infrared light to non-invasively perform: imaging of blood vessels including the blood vessels in the brain; imaging of lymph nodes; and detection of cancer tumors with high sensitivity.

The shortwave infrared fluorescent agent in accordance with an embodiment of the present invention can also be used for drug evaluation with the antibody-drug conjugate (ADC), by, for example, combining a drug components with the molecular recognition agent in the complex described above.

The shortwave infrared fluorescent agent in a case where the agent is used for bioimaging can be administered to an object of imaging by an appropriate known method. For example, the administration of the shortwave infrared fluorescent agent can be direct administration to a part to be imaged or can be intravascular administration to a living body such as an intravenous injection.

### Effects

The compound of the formula (1) in an embodiment of the present invention is an analogue of ICG whose molecular structure is similar to ICG in clinical applications. ICG is currently used as a pharmaceutical agent such as angiography of the retina in humans and liver function testing (ICG testing). The compound of the formula (1) in accordance with the present embodiment has a molecular structure similar to that of ICG. Similarly to ICG, the compound substantially has no cytotoxicity as demonstrated in Examples described later, and can be used in the state of an aqueous solution. Furthermore, the compound of the formula (1) can be used in a form suitable for imaging with shortwave infrared, similarly to conventionally used ICG. For example, the compound of the formula (1) can be bonded to an antibody by a method similar to that in a case of ICG.

Quantum dots each having a final hydrodynamic diameter of less than 5 nm are known to be quickly and efficiently excreted to urine from a living body and removed from the body (see, for example, “Hak Soo Choi1 et al., NATURE BIOTECHNOLOGY VOLUME 25 NUMBER 10 OCTOBER 2007”). Therefore, the compound of the formula (1) in accordance with the present embodiment also has sufficiently high renal clearance for substantial discharge out of the living body. Thus, the compound of the formula (1), which is an analogue of ICG, is highly safe to a living body.

The compound of the formula (1) has a sulfone group. Therefore, the compound is highly water-soluble and is dissolved in a physiological buffer solution. Since the compound is an organic molecule that is highly water-soluble, the compound is, when introduced into a living body, easily discharged from the body.

The compound of the formula (1) can be excited with light having a wavelength in a long wavelength range (900 nm or more) in the near-infrared region. The compound also has shortwave infrared fluorescence intensity that is sufficient for fluorescence imaging of a living body in the state of an aqueous solution. The wavelength region of shortwave infrared is known to have weak absorption and scattering by biological tissues and weak autofluorescence from biological tissues. Therefore, it is possible with the compound of the formula (1) to achieve fluorescence imaging at, in a living body, a part deeper than a part observable by fluorescence imaging with ICG.

In addition, the excitation wavelength of the compound of the formula (1) is at a longer wavelength side in comparison with ICG. Therefore, light absorption of excitation light into biological tissues is made less in comparison with fluorescence imaging in a living body with ICG. Therefore, applying the compound of the formula (1) to fluorescence imaging of a living body makes it possible to further reduce the effect of fluorescence imaging on biological tissues.

Furthermore, in a case where the compound of the formula (1) has a reactive crosslinking group such as an N-hydroxysuccinimide ester group, the reactive crosslinking group can easily bond to a biomolecule such as an antibody. This makes it possible to easily form a complex as a shortwave infrared fluorescence label. Therefore, it is easy to develop an optical contrast medium for molecular imaging, and also possible to perform non-invasive molecular imaging in a shortwave infrared region.

Furthermore, the compound of the formula (1) is capable of causing the exposure time of excitation light to be relatively short. Therefore, by applying the compound of the formula (1) to fluorescence imaging of a living body, it is possible to efficiently perform fluorescence of the compound of the formula (1) and detection of a biological tissue thereof.

Aspects of the present invention can also be expressed as follows:

As is clear from the above description, the compound in accordance with an embodiment of the present invention is represented by the above-described formula (1). This makes it possible to provide a novel compound that enables shortwave infrared bioimaging.

The reactive crosslinking group in the formula (1) can contain at least one organic group selected from the group consisting of an N-hydroxysuccinimide ester group, a maleimide group, an alkynyl group, and an azide group. This feature is more effective from the viewpoint of obtaining a complex with stability and a good yield, as the reactive crosslinking group is bonded to a molecular recognition agent derived from a living body such as an antibody to constitute the complex.

n in the formula (1) can be 3 or 4. This feature is more effective from the viewpoint of bringing about sufficient water-solubility and sufficient fluorescence intensity of shortwave infrared.

The compound of the formula (1) can be a compound represented by one of the above-described formulas (2) to (9). This feature is suitable from the viewpoint of using the compound for bioimaging and molecular imaging with shortwave infrared.

In the complex in accordance with an embodiment of the present invention, the compound in accordance with an embodiment of the present invention is bonded to a molecular recognition agent via a residue of the reactive crosslinking group in the compound. The complex can be used for bioimaging and molecular imaging with shortwave infrared.

In the complex, the molecular recognition agent can be an antibody or a fragment of the antibody with an antigen-binding capacity. In an example, the antibody or a fragment thereof is an antibody or a fragment thereof that specifically bonds to an antigen contained in a biological tissue to be subjected to molecular imaging. This feature is more effective from the viewpoint of achieving molecular imaging in a biological tissue with shortwave infrared.

A shortwave infrared fluorescent agent in accordance with an embodiment of the present invention contains the compound in accordance with an embodiment of the present invention and/or the complex in accordance with an embodiment of the present invention. Therefore, the shortwave infrared fluorescent agent can be used for bioimaging and molecular imaging with shortwave infrared.

A compound production method in accordance with an embodiment of the present invention includes a first step of synthesizing a first compound represented by the above-described formula (1a), a second step of replacing an anilino group in the formula (1a) with a structure represented by the above-described formula (1b), and a third step of replacing a phenylimino group in the formula (1a) with a structure represented by the above-described formula (1c). Therefore, with the production method, it is possible to produce a novel compound (compound of the formula (1)) that enables shortwave infrared bioimaging.

In the compound production method in accordance with an embodiment of the present invention, in a case where the n is 3 and the X is a salt of a sulfonic acid group in the above-described formula, the second step and the third step are carried out simultaneously. This feature is more effective from the viewpoint of more easily producing the compound of the above formula (2) and from the viewpoint of suppressing a decrease in yield due to an increase in the numbers of steps.

In the compound production method in accordance with an embodiment of the present invention, in a case where the n is 4 and the X is a salt of a sulfonic acid group in the above-described formula, the third step can be carried out after the second step. This feature is more effective from the viewpoint of increasing the yield of the replacement of the phenylimino group in the formula (1a) in the third step in the production of the compound of the above formula (3).

In the compound production method in accordance with an embodiment of the present invention, in a case where the n is 3 and the X is a reactive crosslinking group in the above-described formula, the third step can be carried out after the second step. This feature is also more effective from the viewpoint of increasing the yield of the replacement of the phenylimino group in the formula (1a) in the third step in the production of the compound of the above formula (4).

In the compound production method in accordance with an embodiment of the present invention, in a case where the n is 4 and the X is a reactive crosslinking group in the above-described formula, the third step can be carried out before the second step. This feature is more effective from the viewpoint of increasing the yield in the replacements of the anilino groups and the phenylimino group in formula (1a) in the production of the compound of the above formula (5).

The present invention is not limited to the description of the above embodiments, but can be altered in many ways by a person skilled in the art within the scope of the claims. The present invention also encompasses, in its technical scope, any embodiment derived by combining technical means disclosed in differing embodiments.

### EXAMPLES

The following description will discuss Examples in the present invention.

### Source of Reagent Purchase and Compound Identification Method

Commercial solvents and chemical agents used in Examples below were purchased from Sigma-Aldrich Co. LLC, Tokyo Chemical Industry Co., Ltd., and FUJIFILM Wako Pure Chemical Corporation.

The synthesis reaction in Examples below was monitored by thin layer chromatography (TLC) on a silica gel plate (0.2 mm, Merck 60 F-254). In Examples below, column chromatography was performed with use of a silica gel (Wakogel (registered trademark of FUJIFILM Wako Pure Chemical Corporation) N60, spherical, 38 μm to 100 μm).

In Examples below, ¹H NMR (500 MHz) spectrum was measured at 25° C. with use of Varian-Inova-500 NMR device (manufactured by Varian Medical Systems, Inc.). As a solvent for measurement, CDCl₃, CD₃OD, or DMSO-d₆ was used. The chemical shift (δ) was measured in ppm. The binding constant value (J) is indicated by hertz (Hz) with respect to CDCl₃ (δ7.27) or CD₃OD (δ3.31, 4.85), DMSO-d₆ (δ2.50), and tetramethylsilane. The following abbreviations are used for the multiplicity of signals.

s: singlet, d: doublet, t: triplet, q: quartet, m: multiplet, dd: doublet-doublet.

In Examples below, a high-resolution mass spectrum was obtained by Q-Exactive Plus mass spectrometer (Thermo Fisher Scientific Inc., Massachusetts, U.S.).

### Example 1: Synthesis of ICG-C9

In accordance with the following synthesis scheme, ICG-C9, which is a compound in accordance with an embodiment of the present invention, was synthesized.

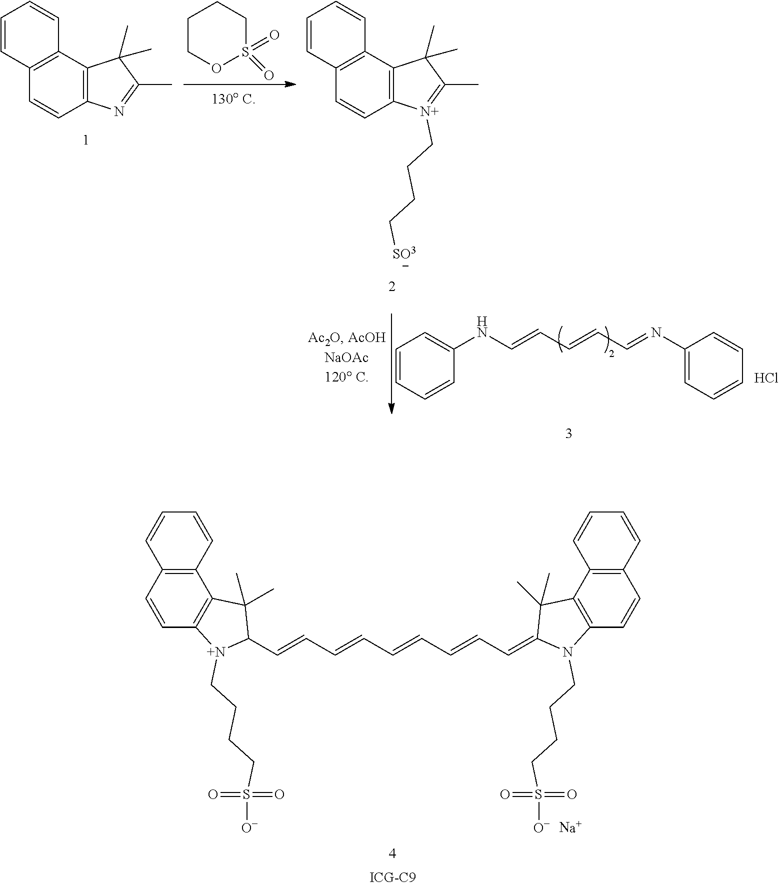

### [Compound 1]

1,1,2-trimethyl-1H-benzo[e]indole (FUJIFILM Wako Pure Chemical Corporation) was prepared as a compound 1.

### [Compound 2]

A mixture of 1,1,2-trimethyl-1H-benzo[e]indole (1) (2 g, 9.6 mmol) and 1,4-butane sulfone (2.6 g, 19.1 mmol) was stirred at 130° C. for 4 hours. The resultant solid was collected by vacuum filtration, and was completely washed with acetone, and dried under vacuum. Thus, a compound 2 was obtained as a light blue solid (2.85 g, yield: 86%). The results of mass spectrometry (MS) and analysis of nuclear magnetic resonance (NMR) of the compound 2 are shown below.

High-Resolution Mass Spectrometry (m/z):
Calculated value: 344.1344
Measured value: 344.131
   with respect to [M−H]⁺ of molecular formula C₁₉H₂₃NO₃S

¹H NMR (DMSO-d₆, 500 MHz): δ=8.37 (1 H, d, J=8.6 Hz), 8.28 (1 H, d, J=9.2 Hz), 8.19-8.21 (2 H, m), 7.77 (1 H, t, J=7.3 Hz), 7.72 (1 H, t, J=7.6 Hz), 4.60 (2 H, t, J=7.8 Hz), 2.94 (3 H, s), 2.51-2.53 (2 H, m), 2.03 (2 H, quintet, J=7.6 Hz), 1.76-1.80 (2 H, m), 1.75 (6 H, s)

### [Compound 3]

The synthesis scheme of a compound 3 is shown below.

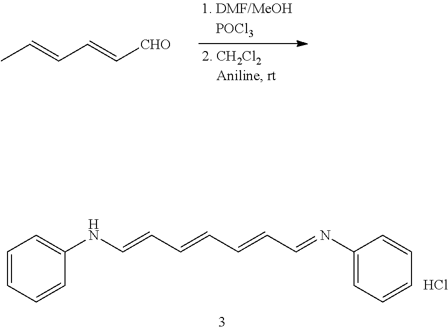

At 0° C., phosphoryl chloride (8 g, 52 mmol) was added by dropping, while stirred was being performed, to a mixed solution of dimethylformamide (10 mL, 130 mmol) and methanol (0.75 mL) under an inert atmosphere. The reaction temperature was raised to 50° C. 2,4-hexadienal 1 (2.5 g, 26 mmol) was dropped, the reaction mixture was stirred at 50° C. for approximately 4 hours, the temperature was cooled to room temperature, and then aqueous sodium perchlorate solution (5% aqueous solution) was added, and extraction was performed with dichloromethane (80 mL). The organic phase was filtered through sodium sulfate. To the dichloromethane phase which was stirred at room temperature, aniline (4.8 g, 52 mmol) was added by dropping, and then the resultant product was stirred overnight. The resultant precipitate was collected by filtration and washed with dichloromethane several times. Thus, the pure compound 3 was obtained as a blue solid (6.8 g, yield: 78%). The results of mass spectrometry (MS) and analysis of nuclear magnetic resonance (NMR) of the compound 3 are shown below.

High-Resolution Mass Spectrometry (m/z):
Calculated value: 275.1542
Measured value: 275.1534
   with respect to [M+H]⁺ of molecular formula C₁₉H₁₈N₂

¹H NMR (CD₃OD, 500 MHz): δ=8.30 (2 H, d, J=11.2 Hz), 7.65 (2 H, t, J=12.6 Hz), 7.42 (4 H, t, J=8.3 Hz), 7.31 (4 H, d, J=7.8 Hz), 7.22 (2 H, t, J=7.8 Hz), 6.49 (7 H, t, J=11.7 Hz), 6.27 (2 H, t, J=11.7 Hz)

### [Compound 4 (ICG-C9)]

At room temperature, sodium acetate (200 mg, 2.42 mmol) was added, while stirring, to a mixed solution of acetic anhydride (10 mL) solution of the compound 2 (0.5 g, 1.45 mmol) and the compound 3 (215 mg, 0.69 mmol). Acetic acid (4 mL) was dropped under an inert atmosphere. The mixture was stirred at 120° C. for 1 hour, and cooled to room temperature. The reaction mixture was transferred to 30 mL of diethyl ether. The resultant precipitate was dissolved in 2-propanol:water (4:1, 50 mL). The resultant product was further diluted with 2-propanol (300 mL). To perform recrystallization, the resultant product was stored at 4° C. overnight. The precipitate was collected by centrifugation, and the resultant residue was further purified by column chromatography, with chloroform/methanol (8:2) serving as an eluate. Thus, ICG-C9, which was a compound 4, was obtained as a brown solid (270 mg, yield: 23%). The results of mass spectrometry (MS) and analysis of nuclear magnetic resonance (NMR) of the compound 4 are shown below.

High-Resolution Mass Spectrometry (m/z):
Calculated value: 777.3026
Measured value: 777.3005

[M]⁺ of molecular formula C₄₅H₄₉N₂O₆S₂

¹H NMR (DMSO-d₆, 500 MHz): δ=8.22 (2 H, d, J=8.3 Hz), 7.89-8.05 (6 H, m), 7.76-7.80 (1 H, m), 7.74 (2 H, d, J=9.3 Hz), 7.59-7.66 (3 H, m), 7.42-7.50 (3 H, m), 6.59 (2 H, t, J=12.7 Hz), 6.42-6.51 (2 H, m), 4.18 (4 H, t, J=6.3 Hz), 1.94-2.02 (4 H, m), 1.90 (12 H, s), 1.71-1.85 (8 H, m), 1.02-1.06 (2 H, m)

### [Cytotoxicity Test of Compound 4]

HeLa cell was seeded on a 96-well plate at 6×10³ per well and was cultured overnight. Aqueous solutions of the compound 4 at concentrations of 0.1 nM, 1 nM, 10 nM and 100 nM were prepared as test agents, and the test agent at each concentration was added to the well, and HeLa cells were further cultured for 6, 24, and 48 hours. After the culturing for each of the above hours, MTT (3-(4,5-di-methylthiazol-2-yl)-2,5-diphenyltetrazolium bromide) reagent was added to the well, and the resultant product was further cultured for 2 hours.

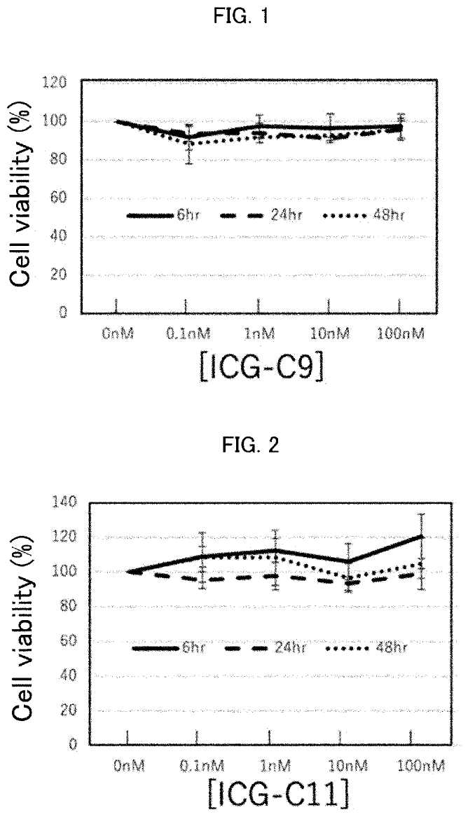

After the culturing for 2 hours, a solubilizing solution was added to the well, and formazan produced in the living cell was dissolved by pipetting. By sealing the 96-well plate and allowing the resultant product to stand in an incubator overnight at 37° C., formazan was completely solubilized. A plate reader was used to measure 570 nm (formazan dye) and 650 nm (background), and the cell viability of the well to which the test agent of each concentration was added was determined, with the cell viability of a well without the addition of a test agent being 100%. Results are shown in FIG. 1. The above test results revealed that, at concentrations of 1 nM to 100 nM, the compound 4 did not exhibit cytotoxicity when cultured for 6 hours, 24 hours, or 48 hours.

### Example 2: Synthesis of ICG-C11

In accordance with the following synthesis scheme, ICG-C11, which is a compound in accordance with an embodiment of the present invention, was synthesized.

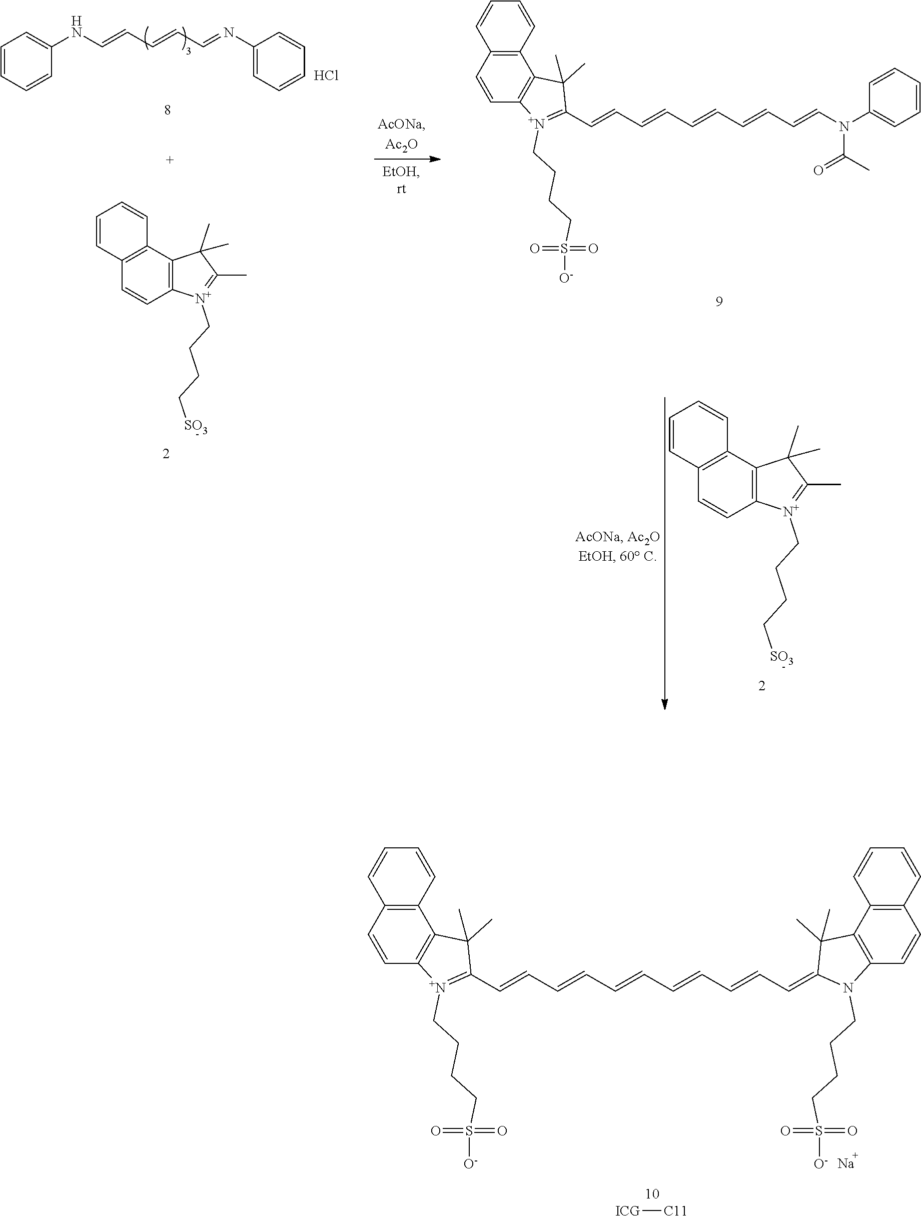

The synthesis scheme of a compound 8 is shown below.

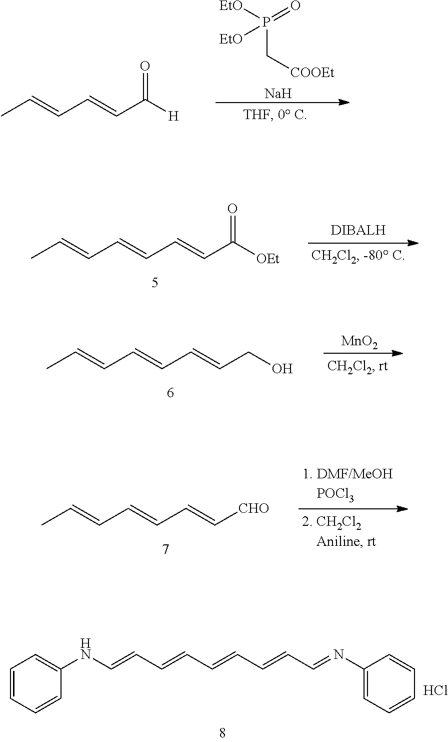

### [Compound 5]

Under an inert atmosphere, triethyl phosphonoacetic acid (5.2 g, 22.1 mmol) was dropped, while stirring, onto a solution of tetrahydrofuran of sodium hydride (60%, 833 mg, 20.8 mmol in mineral oil) at 0° C. The mixture was stirred at 0° C. for 30 minutes, and then 2,4-hexadienal 1 (2 g, 20.8 mmol) was added by dropping. The reactant was warmed to room temperature, stirred for approximately 2 hours, and quenched with ammonium chloride, and then extraction was performed with ethyl acetate. The organic phase was dried with sodium sulfate, filtered, and concentrated in a vacuum, so that a residue was obtained. The residue was purified by column chromatography with hexane/ethyl acetate (9.5:0.5) serving as an eluate. Thus, a compound 5 was obtained as a white solid (3.25 g, yield: 94%). The results of mass spectrometry (MS) and analysis of nuclear magnetic resonance (NMR) of the compound 5 are shown below.

High-Resolution Mass Spectrometry (m/z):
Calculated value: 167.1066
Measured value: 167.1068
   with respect to [M+H]⁺ of molecular formula C₁₀H₁₄O₂

¹H NMR (CDCl₃, 500 MHz): δ=7.31 (1 H, dd, J=11.3, 4.4 Hz), 6.53 (1 H, dd, J=10.7, 4.4 Hz), 6.14-6.24 (2 H, m), 5.90-5.98 (1 H, m), 5.86 (1 H, d, J=15.1 Hz), 4.21 (2 H, m), 1.34 (3 H, d, J=6.4 Hz), 1.30 (3 H, t, J=7.5 Hz)

### [Compound 6]

Under an inert atmosphere, while a dichloromethane solution (2.5 g, 15 mmol) of the compound 5 was stirred at −60° C., diisobutylaluminum hydride (1.0 M, 45 mL, 45 mmol in hexane) was dropped. The reactant was stirred at −60° C. for 30 minutes, and then at 0° C. for 1 hour. Water (10 mL) was slowly added, and subsequently sodium hydroxide (10 mL of 15% aqueous solution) was added and the reaction was quenched, so that a white precipitate was obtained. The mixture was vigorously stirred at room temperature for 1 hour, magnesium sulfate was added, and the mixture was filtered by a Celite containing dichloromethane. The organic phase was concentrated in a vacuum, so that a crude was obtained. The crude was purified by column chromatography with hexane/ethyl acetate (8:2) serving as an eluate. Thus, a compound 6 was obtained as a white solid (1.65 g, yield: 89%). The results of mass spectrometry (MS) and analysis of nuclear magnetic resonance (NMR) of the compound 6 are shown below.

High-Resolution Mass Spectrometry (m/z):
Calculated value: 107.0855
Measured value: 107.0860
   with respect to [M+H−H₂O]⁺ of molecular formula C₈H₁₀O

¹H NMR (CDCl₃, 500 MHz): δ=6.20-6.28 (2 H, m), 6.06-6.14 (2 H, m), 5.71-5.83 (2 H, m), 4.20 (2 H, d, J=5.8 Hz), 1.79 (2 H, d, J=6.8 Hz), 1.40 (1 H, s)

### [Compound 7]

Under an inert atmosphere, at room temperature, activated manganese oxide (7.4 g, 84.7 mmol) was added to a dichloromethane solution (30 mL) of the compound 6 (1.5 g, 12.1 mmol). The reaction mixture was stirred overnight. The reaction mixture was filtered, and the filtrate was concentrated. With hexane/ethyl acetate (9:1) serving as an eluate, a purified crude was obtained by column chromatography. Thus, a compound 7 was obtained as a yellow solid (1.08 g, yield: 73%). The results of mass spectrometry (MS) and analysis of nuclear magnetic resonance (NMR) of the compound 7 are shown below.

High-Resolution Mass Spectrometry (m/z):
Calculated value: 123.0804
Measured value: 123.0804
   with respect to [M+H]⁺ of molecular formula C₈H₁₀O

¹H NMR (CDCl₃, 500 MHz): δ=9.58 (1 H, d, J=9.8 Hz), 7.12 (1 H, dd, J=11.3, 3.9 Hz), 6.65 (1 H, dd, J=10.3, 4.4 Hz), 6.34 (1 H, dd, J=10.7, 4.0 Hz), 6.21 (1 H, m), 6.14 (1 H, dd, J=7.8, 7.3 Hz), 6.05 (1 H, m), 1.87 (3 H, d, J=7.8 Hz)

### [Compound 8]

At 0° C., phosphoryl chloride (2.5 g, 16.4 mmol) was added by dropping, while stirred was being performed, to a solution of dimethylformamide (3.2 mL, 42.5 mmol) and methanol (0.4 mL) under an inert atmosphere. The reaction temperature was raised to 40° C. A dimethylformamide solution (0.5mL) of the compound 7 (1 g, 8.2 mmol) was dropped, and the reaction mixture was stirred at 40° C. for approximately 4 hours. The reactant was cooled to room temperature, and then added to an aqueous sodium perchlorate solution (1 g in 20 mL), and then extraction was performed with dichloromethane (25 mL). The organic phase was filtered through a sodium sulfate layer. To the dichloromethane phase which was stirred at room temperature, aniline (1.5 g, 16.4 mmol) was dropped, and then the resultant product was stirred overnight. The resultant precipitate was collected by filtration and washed with dichloromethane several times. Thus, a compound 8 was obtained as a green solid (1.25 g, yield: 45%). The results of mass spectrometry (MS) and analysis of nuclear magnetic resonance (NMR) of the compound 8 are shown below.

High-Resolution Mass Spectrometry (m/z):
Calculated value: 301.1699
Measured value: 301.1699
   with respect to [M+H]⁺ of molecular formula C₂₁H₂₀N₂

¹H NMR (CD₃OD, 500 MHz): δ=8.21 (2 H, d, J=11.2 Hz), 7.52 (2 H, t, J=13.2 Hz), 7.4 (4 H, t, J=8.3 Hz), 7.367 (1 H, d, J=12.7 Hz), 7.27 (4 H, d, J=7.8 Hz), 7.18 (2 H, t, J=7.3 Hz), 6.47 (2 H, t, J=13.2 Hz), 6.26 (2 H, t, J=11.7 Hz)

### [Compound 9]

At room temperature, to an ethanol (10 mL) solution of the compound 2 (250 mg, 0.72 mmol) and sodium acetate (178 mg, 2.17 mmol), while the solution was being stirred, the compound 8 (244 mg, 0.72 mmol) was added. Next, acetic anhydride (0.4 mL, 4.34 mmol) was dropped under an inert atmosphere. The reaction was continued for 30 minutes, and the solvent was removed in a vacuum. The residue was purified by column chromatography with chloroform/methanol (9:1) serving as an eluate. Thus, a compound 9 was obtained as a gold-brown solid (274 mg, yield: 64%). The results of mass spectrometry (MS) and analysis of nuclear magnetic resonance (NMR) of the compound 9 are shown below.

High-Resolution Mass Spectrometry (m/z):
Calculated value: 595.2625
Measured value: 595.2617
   with respect to [M+H]⁺ of molecular formula C₃₆H₃₈N₂O₄S

¹H NMR (CD₃OD, 500 MHz): δ=8.35 (1 H, d, J=7.3 Hz), 8.20-8.25 (2 H, m), 8.11 (1 H, d, J=8.3 Hz), 7.86-7.94 (2 H, m), 7.76 (1 H, t, J=7.3 Hz), 7.65 (1 H, t, J=7.8 Hz), 7.53-7.61 (3 H, m), 7.28-7.36 (3 H, m), 7.12 (1 H, d, J=14.6 Hz), 6.86 (2 H, q, J=14.1 Hz), 6.69 (1 H, t, J=11.2 Hz), 6.49 (1 H, t, J=13.6 Hz), 6.20 (1 H, t, J=11.7 Hz), 5.23 (2 H, t, J=13.7 Hz), 4.59 (2 H, t, J=7.3 Hz), 2.9 (2 H, t, J=7.3 Hz), 2.14 (2 H, quintet, J=7.8 Hz), 2.00 (6 H, s), 1.89-1.98 (2 H, m)

### [Compound 10 (ICG-C11)]

At room temperature, to an ethanol (3 mL) solution of the compound 2 (29 mg, 0.084 mmol) and sodium acetate (12 mg, 0.139 mmol), while the solution was being stirred, acetic anhydride (9 μL, 0.092 mmol) was added. The compound 9 (50 mg, 0.084 mmol) dissolved in ethanol (3 mL) was added to the reaction mixture under an inert atmosphere. The mixture was stirred at 60° C. for 1 hour. The solvent was removed in a vacuum, and the residue was purified by column chromatography using chloroform/methanol (7.5:2.5). Thus, ICG-C11, which was a compound 10, was obtained as a brown solid (33 mg, yield: 48%). The results of mass spectrometry (MS) and analysis of nuclear magnetic resonance (NMR) of the compound 10 are shown below.

High-Resolution Mass Spectrometry (m/z):
Calculated value: 803.3183
Measured value: 803.3221

[M]⁺ of molecular formula C₄₇H₅₁N₂O₆S₂

¹H NMR (DMSO-d₆, 500 MHz): δ=8.21 (2 H, d, J=8.3 Hz), 8.01 (4 H, d, J=7.3 Hz), 7.81 (2 H, t, J=13.1 Hz), 7.71 (2 H, t, J=8.8 Hz), 7.61 (2 H, t, J=6.8 Hz), 7.47 (2 H, t, J=7.3 Hz), 7.34 (2 H, t, J=12.7 Hz), 7.01 (1 H, t, J=12.7 Hz), 6.48-6.57 (4 H, m), 6.44 (2 H, d, J=13.6 Hz), 4.17 (4 H, t, J=6.8 Hz), 1.88 (12 H, s), 1.75-1.81 (12 H, m)

### [Cytotoxicity Test of Compound 10]

Culturing of HeLa cells and determination of cell viability of the well were performed as in the cytotoxicity test for the compound 4, except that a test agent was prepared using the compound 10 instead of the compound 4. Results are shown in FIG. 2. The above test results revealed that, at concentrations of 1 nM to 100 nM, the compound 10 as in the case of the compound 4 did not exhibit cytotoxicity when cultured for 6 hours, 24 hours, or 48 hours.

### Example 3: Synthesis of ICG-C9-NHS

In accordance with the following synthesis scheme, ICG-C9-NHS, which is a compound in accordance with an embodiment of the present invention, was synthesized.

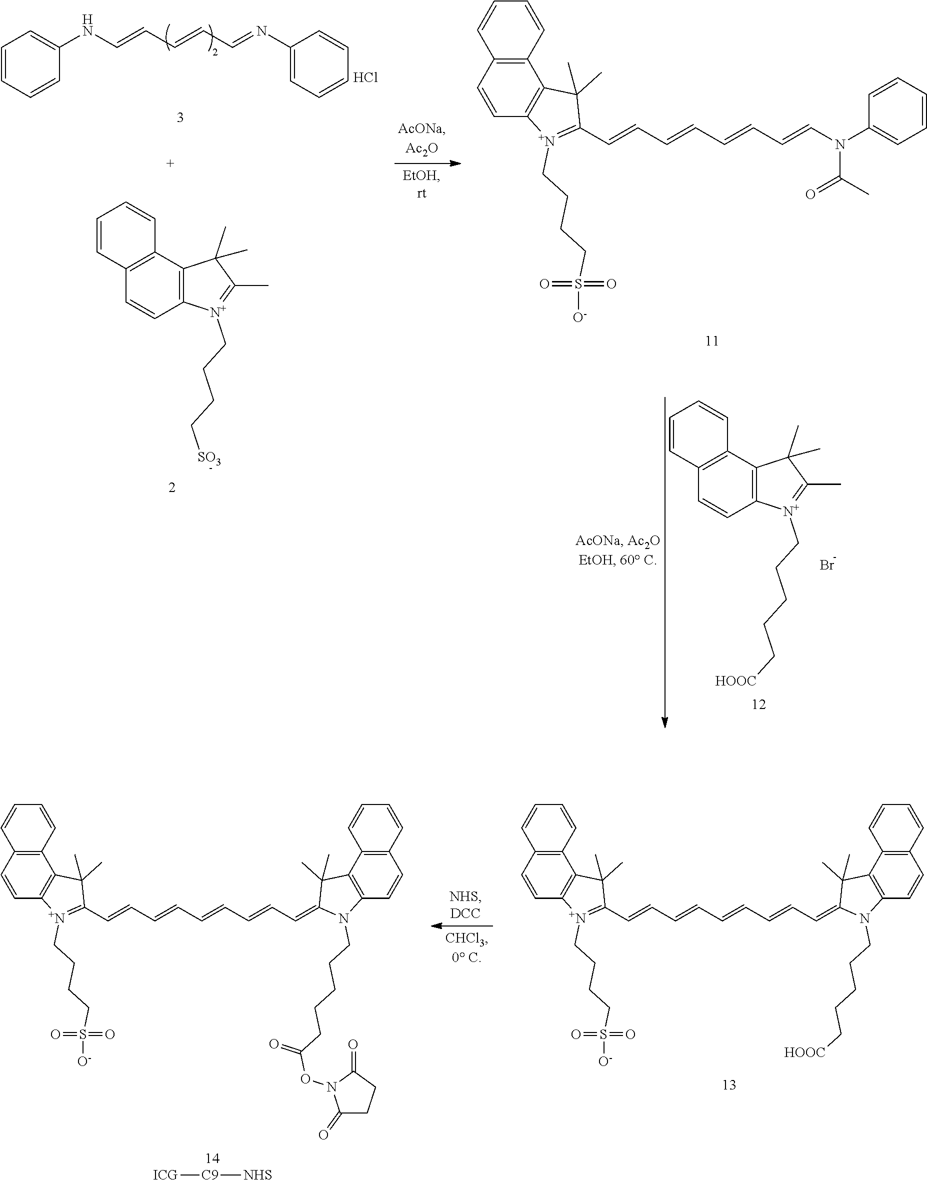

### [Compound 11]

At room temperature, to a mixed solution of the compound 2 (1 g, 2.89 mmol), sodium acetate (712 mg, 8.68 mmol), and ethanol (25 mL), the compound 3 (900 mg) was added while the solution was being stirred. Next, acetic anhydride (1.7 mL, 17.37 mmol) was dropped under an inert atmosphere. The reaction was continued for 30 minutes, and the solvent was removed in a vacuum. The residue was purified by column chromatography with chloroform/methanol (9:1) serving as an eluate. Thus, a compound 11 was obtained as a gold-brown solid (1.12 g, yield, 68%). The results of mass spectrometry (MS) and analysis of nuclear magnetic resonance (NMR) of the compound 11 are shown below.

High-Resolution Mass Spectrometry (m/z):
Calculated value: 569.2468
Measured value: 569.2481
   with respect to [M+H]⁺ of molecular formula C₃₄H₃₆N₂OS

¹H NMR (CD₃OD, 500 MHz): δ=8.35 (1 H, d, J=8.3 Hz), 8.22 (1 H, dd, J=11.25, 3.4 Hz), 8.18 (1 H, d, J=8.8 Hz), 8.11 (1 H, d, J=8.3 Hz), 8.00-8.04 (1 H, m), 7.93 (1 H, d, J=8.7 Hz), 7.76 (1 H, t, J=7.3 Hz), 7.56-7.67 (4 H, m), 7.31-7.36 (3 H, m), 7.07 (1 H, d, J=15.1 Hz), 6.95 (1 H, dd, J=11.3 Hz, 3.0 Hz), 6.75 (1 H, dd, J=11.3, 3.0 Hz), 6.33 (1 H, dd, J=11.7, 2.5 Hz), 5.30 (1 H, dd, J=11.7 Hz, 2.0 Hz), 4.58 (2 H, t, J=7.8 Hz), 2.90 (2 H, t, J=7.3 Hz), 2.10-2.16 (2 H, m), 2.00 (6 H, s), 1.98 (3 H, s), 1.95-1.97 (2 H, m)

### [Compound 12]

The synthesis scheme of a compound 12 is shown below.

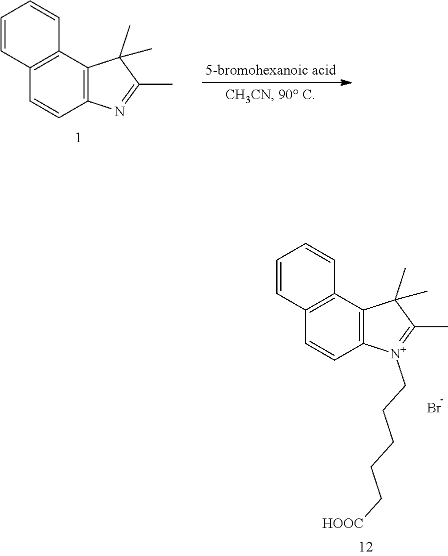 [SMILES: CC1=Nc2ccc3ccccc3c2C1(C)C ; CC1=[N+](CCCCCC(=O)O)c2ccc3ccccc3c2C1(C)C ; Br]

An acetonitrile solution (100 mL) of 1,1,2-trimethyl-1H-benzo[e]indole (compound 1) (2.5 g, 11.9 mmol) and 6-bromohexanoic acid (7 g, 35.8 mmol) was stirred under an inert atmosphere at 90° C. for 2 days. The solvent was removed in a vacuum, and the residue was dissolved in dichloromethane (25 mL). The resultant product was diluted with diethyl ether, and a product was obtained as a precipitate. The product was filtered and washed with diethyl ether. Thus, a compound 12 was obtained as a light green solid (3.8 g, yield: 79%). The results of mass spectrometry (MS) and analysis of nuclear magnetic resonance (NMR) of the compound 12 are shown below.

High-Resolution Mass Spectrometry (m/z):
Calculated value: 324.1958
Measured value: 324.1949
   with respect to [M]⁺ of molecular formula C₂₁H₂₆NO₂

¹H NMR (DMSO-d₆, 500 MHz): δ=8.38 (1 H, d, J=8.8 Hz), 8.29 (1 H, d, J=8.8 Hz), 8.22 (1 H, d, J=7.9 Hz), 8.15 (1 H, d, J=8.8 Hz), 7.78 (1 H, t, J=7.4 Hz), 7.73 (1 H, t, J=6.9 Hz), 4.57 (2 H, t, J=7.8 Hz), 2.93 (3 H, s), 2.23 (2 H, t, J=7.3), 1.87-1.93 (2 H, m), 1.75 (6 H, s), 1.54-1.60 (2 H, m), 1.43-1.49 (2 H, m)

### [Compound 13]

At room temperature, to a mixed solution of the compound 12 (164 mg, 0.405 mmol), sodium acetate (55 mg, 0.673 mmol), and ethanol (8 mL), acetic anhydride (43 μL, 0.446 mmol) was added while the solution was being stirred. The compound 11 (230 mg, 0.405 mmol) dissolved in ethanol (10 mL) was added to the reaction mixture under an inert atmosphere. The mixture was stirred at 60° C. for 1 hour. The solvent was removed in a vacuum. The residue was purified by column chromatography with chloroform/methanol (8:2) serving as an eluate. Thus, a compound 13 was obtained as a dark green solid (234 mg, yield: 76%). The results of mass spectrometry (MS) and analysis of nuclear magnetic resonance (NMR) of the compound 13 are shown below.

High-Resolution Mass Spectrometry (m/z):
Calculated value: 757.3669
Measured value: 757.3666
   with respect to [M+H]⁺ of molecular formula C₄₇H₅₂N₂O₅S

¹H NMR (CD₃OD, 500 MHz): δ=8.20 (2 H, t, J=7.8 Hz), 7.85-8.00 (7 H, m), 7.58-7.65 (3 H, m), 7.52 (1 H, d, J=9.3 Hz), 7.34-7.48 (4 H, m), 6.50-6.64 (3 H, m), 6.42 (1 H, d, J=13.6 Hz), 6.25 (1 H, d, J=13.1 Hz), 4.24 (2 H, t, J=6.3 Hz), 4.16 (2 H, t, J=6.8 Hz), 3.61 (1 H, q, t, J=6.8 Hz), 2.92 (2 H, t, J=6.8 Hz), 2.29 (2 H, t, J=7.3 Hz), 1.99-2.07 (4 H, m), 1.86 (2 H, quintet, J=7.3 Hz), 1.71 (2 H, quintet, J=7.3 Hz), 1.53 (2 H, quintet, J=7.3 Hz)

### [Compound 14 (ICG-C9-NHS)]

Under an inert atmosphere, to a chloroform solution (10 mL) of the compounds 13 (200 mg, 0.264 mmol) and N-hydroxysuccinimide (76 mg, 0.660 mmol), while the solution was being stirred, a chloroform solution (1 mL) of N,N′-dicyclohexylcarbodiimide (136 mg, 0.660 mmol) was dropped. The reaction mixture was stirred at room temperature for 4 hours. The solvent was removed in a vacuum, and the residue was purified by column chromatography, using chloroform/methanol (9:1) as an eluate. Thus, ICG-C9-NHS, which was a compound 14, was obtained as a dark green solid (125 mg, yield: 55%). The results of mass spectrometry (MS) and analysis of nuclear magnetic resonance (NMR) of the compound 14 are shown below.

High-Resolution Mass Spectrometry (m/z):
Calculated value: 854.3833
Measured value: 854.3828
   with respect to [M+H]⁺ of molecular formula C₅₁H₅₅N₃O₇S

¹H NMR (CD₃OD, 500 MHz): δ=8.21 (2 H, t, J=7.3 Hz), 7.96 (6 H, m), 7.62 (3 H, m), 7.53 (1 H, d, J=8.8 Hz), 7.34-7.53 (4 H, m), 6.50-6.65 (3 H, m), 6.42 (1 H, d, J=13.6 Hz), 6.27 (1 H, d, J=13.1 Hz), 4.24 (2 H, t, J=6.3 Hz), 1.46 (2 H, t, J=7.3 Hz), 2.92 (2 H, t, J=7.3 Hz), 2.65-2.70 (2 H, m), 2.00-2.07 (4 H, m), 1.97 (12 H, s), 1.82-1.92 (6 H, m), 1.61-1.73 (2 H, m), 1.39-1.45 (2 H, m)

### Example 4: Synthesis of ICG-C11-NHS

In accordance with the following synthesis scheme, ICG-C11-NHS, which is a compound in accordance with an embodiment of the present invention, was synthesized.

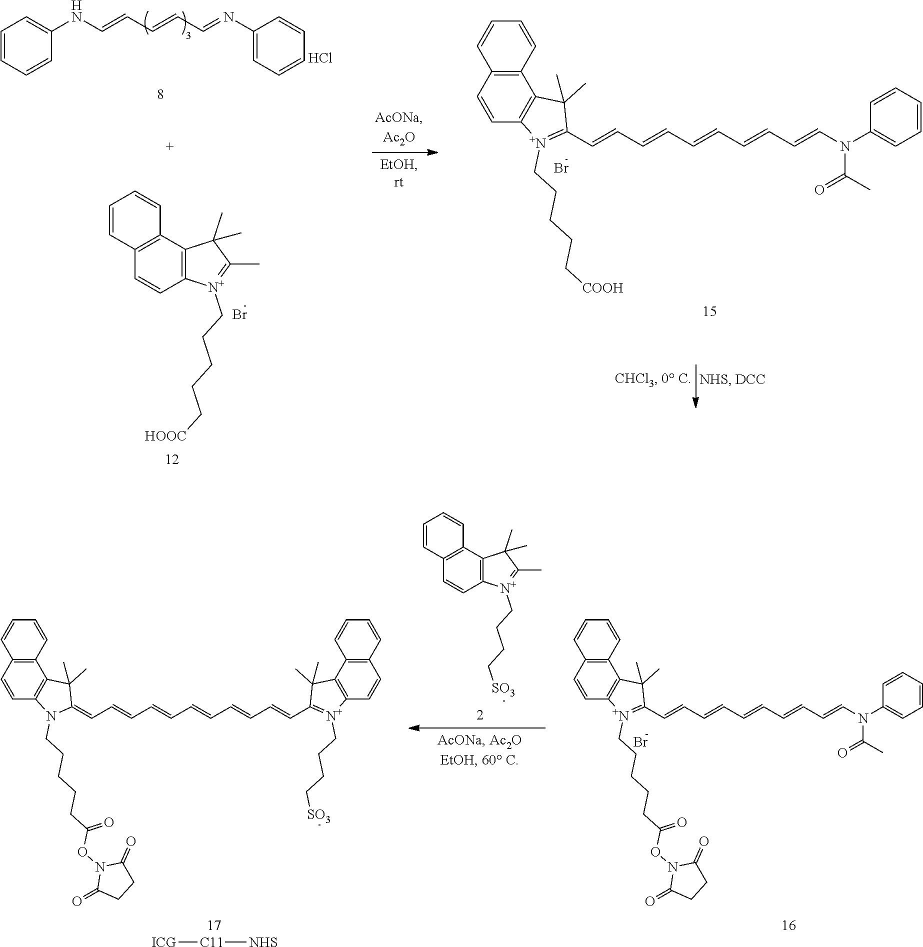

### [Compound 15]

At room temperature, to an ethanol solution (15 mL) of the compound 12 (250 mg, 0.618 mmol) and sodium acetate (101 mg, 1.236 mmol), the compound 8 (208 mg, 0.618 mmol) was added while the solution was being stirred. Next, acetic anhydride (175 μL, 1.855 mmol) was dropped under an inert atmosphere. The reaction was continued for 30 minutes, and the solvent was removed in a vacuum. The residue was purified by column chromatography with chloroform/methanol (9:1) serving as an eluate. Thus, a compound 15 was obtained as a gold-brown solid (310 mg, yield: 77%). The results of mass spectrometry (MS) and analysis of nuclear magnetic resonance (NMR) of the compound 15 are shown below.

High-Resolution Mass Spectrometry (m/z):
Calculated value: 573.3111
Measured value: 573.3108
   with respect to [M]⁺ of molecular formula C₃₈H₄₁N₂O₃

¹H NMR (CD₃OD, 500 MHz): δ=8.36 (1 H, d, J=8.3 Hz), 8.25 (1 H, dd, J=11.2, 4.0 Hz), 8.19 (1 H, d, J=8.8 Hz), 8.12 (1 H, d, J=8.3 Hz), 7.89 (2 H, m), 7.77 (1 H, t, J=7.3 Hz), 7.67 (1 H, t, J=7.3 Hz), 7.60 (2 H, j, J=7.4 Hz), 7.54 (1 H, t, J=7.8 Hz), 7.36 (1 H, dd, J=11.8, 2.4 Hz), 7.30 (2 H, d, J=7.4 Hz), 7.04 (1 H, d, J=15.1 Hz), 6.79-6.90 (2 H, m), 6.71 (1 H, dd, J=11.3, 3.4 Hz), 6.50 (1 H, dd, J=11.3, 3.0 Hz), 6.21 (1 H, dd, J=11.3, 3.4 Hz), 5.23 (1 H, dd, J=11.3, 3.0 Hz), 4.56 (2 H, t, J=7.3 Hz), 2.29 (2 H, t, J=7.3 Hz)2.01 (6 H, s), 1.92-1.97 (5 H, m), 1.69 (2 H, quintet, J=6.8 Hz), 1.54 (2 H, quintet, J=7.3 Hz)

### [Compound 16]

Under an inert atmosphere, at 0° C., to a chloroform solution (10 mL) of the compounds 15 (110 mg, 0.168 mmol) and N-hydroxysuccinimide (48 mg, 0.42 mmol), while the solution was being stirred, a chloroform solution (1 mL) of N,N′-dicyclohexylcarbodiimide (86 mg, 0.42 mmol) was dropped. The reaction mixture was stirred at room temperature for 2 hours. The solvent was removed in a vacuum. The residue was purified by column chromatography with chloroform/methanol (9.5:0.5) serving as an eluate. Thus, a compound 16 was obtained as a brown solid (88 mg, yield: 70%). The results of mass spectrometry (MS) and analysis of nuclear magnetic resonance (NMR) of the compound 16 are shown below.

High-Resolution Mass Spectrometry (m/z):
Calculated value: 670.3275
Measured value: 670.3272
   with respect to [M]⁺ of molecular formula C₄₂H₄₄N₃O₅

¹H NMR (CD₃OD, 500 MHz): δ=8.36 (1 H, d, J=8.3 Hz), 8.24 (1 H, t, J=11.7 Hz), 8.18 (1 H, d, J=9.3 Hz), 8.12 (1 H, d, J=7.8 Hz), 7.87-7.89 (2 H, m), 7.77 (1 H, t, J=7.3 Hz), 7.67 (1 H, t, J=7.8 Hz), 7.54-7.61 (4 H, m), 7.29-7.38 (3 H, m), 7.04 (1 H, d, J=14.6 Hz), 6.78-6.90 (2 H, m), 6.72 (1 H, t, J=13.6 Hz), 6.50(H, t, J=11.2 Hz), 6.21 (1 H, t, J=14.1 Hz), 5.24 (1 H, t, J=11.7 Hz), 4.57 (2 H, t, J=6.3 Hz), 2.76-2.84 (4 H, m), 2.01 (6 H, s), 1.89-1.97 (4 H, m), 1.85 (2 H, quintet, J=6.8 Hz), 1.64 (2 H, quintet, J=5.0 Hz)

### [Compound 17 (ICG-C11-NHS)]

At room temperature, acetic anhydride (15 μL, 0.160 mmol) was added to an ethanol solution (3 mL) of the compounds 2 (55 mg, 0.160 mmol) and sodium acetate (13 mg, 0.160 mmol) while the solution was being stirred. The compound 16 (80 mg, 0.106 mmol) dissolved in ethanol (5 mL) was added to the reaction mixture under an inert atmosphere. The mixture was stirred at 60° C. for 1 hour. The solvent was removed in a vacuum. The residue was purified by column chromatography with chloroform/methanol (9:1) serving as an eluate. Thus, ICG-C11-NHS, which was a compound 17, was obtained as a dark green solid (42 mg, yield: 45%). The results of mass spectrometry (MS) and analysis of nuclear magnetic resonance (NMR) of the compound 17 are shown below.

High-Resolution Mass Spectrometry (m/z):
Calculated value: 880.3990
Measured value: 880.3985
   with respect to [M+H]⁺ of molecular formula C₅₃H₅₇N₃O₇S

¹H NMR (DMSO-d₆, 500 MHz): δ=8.25 (1 H, d, J=8.3 Hz), 8.18 (1 H, d, J=8.8 Hz), 8.06 (2 H, t, J=8.8 Hz), 7.99 (2 H, d, J=8.8 Hz), 7.92 (1 H, t, J=12.7 Hz), 7.80 (1 H, d, J=8.8 Hz), 7.71 (1 H, t, J=12.7 Hz), 7.65 (1 H, t, J=7.3 Hz), 7.60 (2 H, t, J=8.8 Hz), 7.51 (1 H, t, J=7.8 Hz), 7.37-7.44 (2 H, m), 7.27 (1 H, t, J=12.7 Hz), 7.01 (1 H, t, J=13.1 Hz), 6.45-6.64 (5 H, m), 6.22 (1 H, d, J=13.7 Hz), 4.25 (2 H, t, J=6.8 Hz), 4.09 (2 H, t, J=7.3 Hz), 2.80 (4 H, s), 2.68 (2 H, t, J=6.8 Hz), 1.90 (6 H, s), 1.88 (6 H, s), 1.82-1.86 (3 H, m), 1.68-1.79 (7 H, m), 1.50 (2 H, quintet, J=7.3 Hz), 1.40 (2 H, quintet, J=6.8 Hz)

### [Another Method for Synthesizing ICG-C11-NHS]

It should be noted that ICG-C11-NHS can also be synthesized in accordance with the synthesis scheme shown below. The following description will discuss another method for synthesizing ICG-C11-NHS in detail.

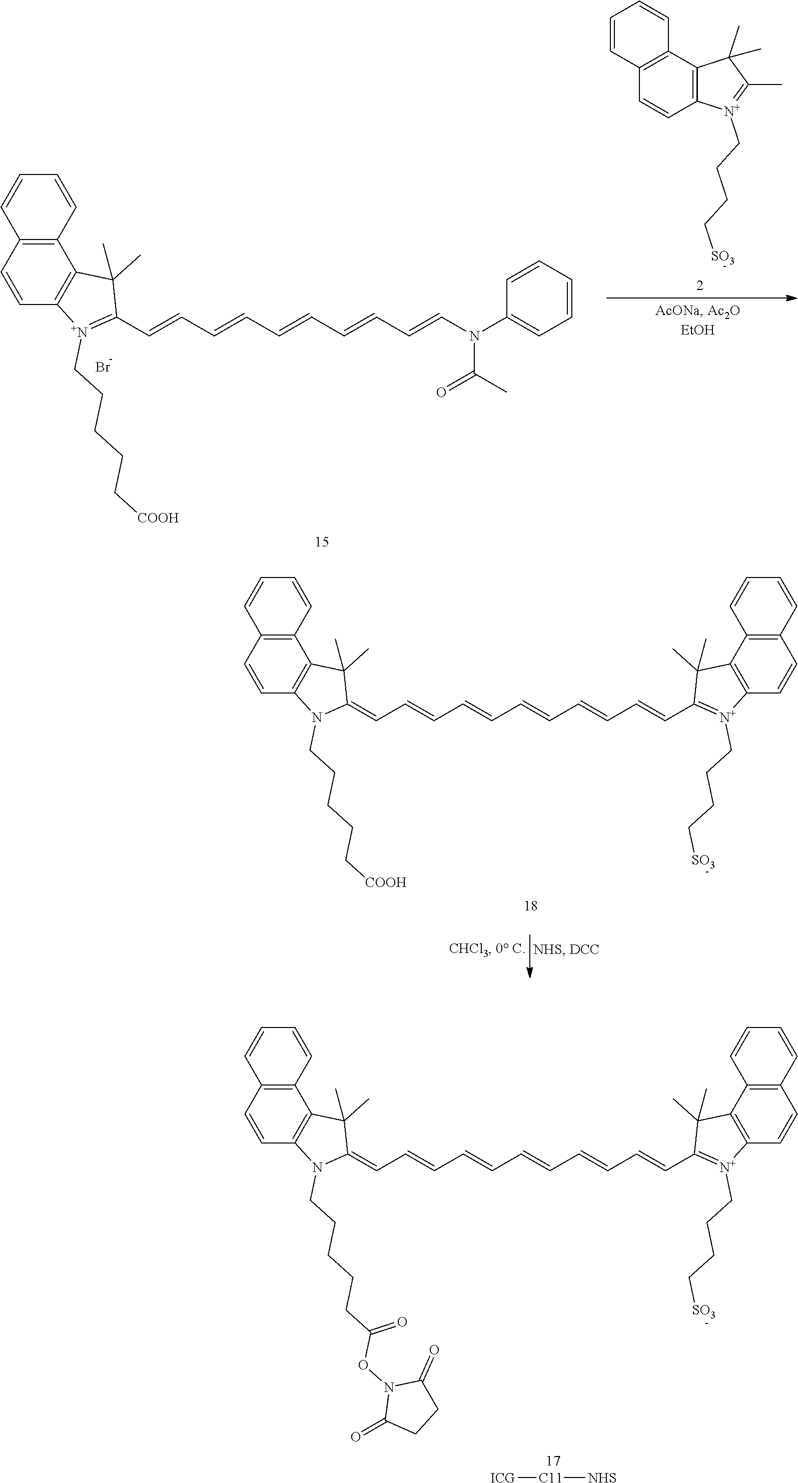

### [Compound 18]

At room temperature, acetic anhydride (53 μL, 0.555 mmol) was added to an ethanol solution (10 mL) of the compounds 2 (175 mg, 0.505 mmol) and sodium acetate (21 mg, 0.838 mmol). Next, the compound 15 (330 mg, 0.505 mmol) dissolved in ethanol (10 mL) was added to the reaction mixture under an inert atmosphere, and the mixture was stirred at 60° C. for approximately 1.5 hours. The solvent was removed in a vacuum, and the residue was purified by column chromatography, using chloroform/methanol (8:2) as an eluate. Thus, a compound 18 was obtained as a dark brown solid (127 mg, 64%). The results of mass spectrometry (MS) and analysis of nuclear magnetic resonance (NMR) of the compound 18 are shown below.

High-Resolution Mass Spectrometry (m/z):
Calculated value: 783.3826
Measured value: 783.3817
   with respect to [M+H]⁺ of molecular formula C₄₉H₅₄N₂O₅S

¹H NMR (DMSO-d₆, 500 MHz): δ=8.25 (1 H, d, J=8.8 Hz), 8.19 (1 H, d, J=8.8 Hz), 8.06 (2 H, t, J=8.3 Hz), 8.00 (2 H, d, J=8.8 Hz), 7.92 (1 H, t, J=12.7 Hz), 7.80 (1 H, d, J=8.8 Hz), 7.71 (1 H, t, J=12.7 Hz), 7.65 (1 H, t, J=7.3 Hz), 7.60 (2 H, t, J=8.8 Hz), 7.51 (1 H, t, J=7.3 Hz), 7.37-7.44 (2 H, m), 7.27 (1 H, t, J=12.7 Hz), 7.01 (1 H, t, J=12.7 Hz), 6.45-6.63 (5 H, m), 6.22 (1 H, d, J=13.1 Hz), 4.24 (2 H, t, J=6.3 Hz), 4.09 (2 H, t, J=6.8 Hz), 2.14 (2 H, t, J=6.8 Hz), 1.90 (6 H, s), 1.88 (6 H, s), 1.82-1.84 (2 H, m), 1.68-1.79 (4 H, m), 1.55 (2 H, quintet, J=7.3 Hz), 1.40 (2 H, quintet, J=6.8 Hz)

### [Compound 17 (ICG-C11-NHS)]

At 0° C. under an inert atmosphere, to chloroform solution (8 mL) of the compounds 18 (53 mg, 0.0677 mmol) and N-hydroxysuccinimide (20 mg, 0.169 mmol), while the solution was being stirred, a chloroform solution (1 mL) of N,N′-dicyclohexylcarbodiimide (35 mg, 0.169 mmol) was dropped. Then, the reaction mixture was stirred at room temperature for 4 hours. The solvent was removed in a vacuum, and the residue was purified by column chromatography, using chloroform/methanol (9:1) as an eluate. Thus, ICG-C11-NHS (compound 17) was obtained as a dark brown solid (31 mg, 52%). The results of mass spectrometry (MS) and analysis of nuclear magnetic resonance (NMR) of the compound 17 obtained are as described above.

### Example 5: Synthesis of ICG-C9-Maleimide

In accordance with the following synthesis scheme, ICG-C9-maleimide, which is a compound in accordance with an embodiment of the present invention, was synthesized.

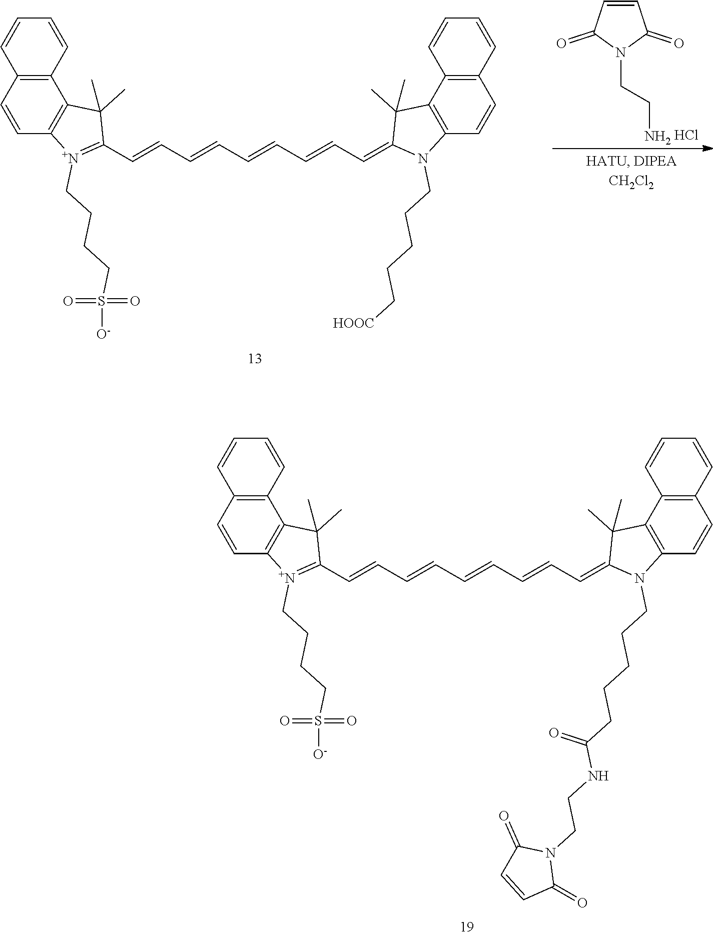 [SMILES: CC1(C)C(/C=C/C=C/C=C/C=C/C=C2/N(CCCCCC(=O)O)c3ccc4ccccc4c3C2(C)C)=[N+](CCCCS(=O)(=O)[O-])c2ccc3ccccc3c21 ; NCCN1C(=O)C=CC1=O ; Cl ; CC1(C)C(/C=C/C=C/C=C/C=C/C=C2/N(CCCCCC(=O)NCCN3C(=O)C=CC3=O)c3ccc4ccccc4c3C2(C)C)=[N+](CCCCS(=O)(=O)[O-])c2ccc3ccccc3c21]

### [Compound 19]

Under an inert atmosphere, at room temperature, 1-[bis(dimethylamino)methylene]-1H-1,2,3-triazolo[4,5-b]pyridinium 3-oxide hexafluorophosphate (HATU) (75 mg, 0.198 mmol), N,N-diisopropylethylamine (DIPEA) (31 mg, 0.237 mmol), and 1-(2-aminoethyl)maleimide hydrochloride (21 mg, 0.119 mmol) were added to a stirred solution of the compound 13 (60 mg, 0.079 mmol) in dichloromethane (10 mL). The reaction mixture was stirred at room temperature for approximately 4 hours to 5 hours. The solvent was removed in a vacuum, and the residue was purified by column chromatography using chloroform/methanol (9:1), so that a compound 19 (45 mg, 64%) was obtained as a dark brown solid. The results of mass spectrometry (MS) and analysis of nuclear magnetic resonance (NMR) of the compound 19 are shown below.

High-Resolution Mass Spectrometry (m/s):
Calculated value: 879.4149
Measured value: 879.4121
   with respect to with respect to [M+H]⁺ of molecular formula C₅₃H₅₈N₄O₆S

¹H NMR (DMSO-d₆, 500 MHz): δ=8.56 (1 H, d, J=3.4 Hz), 8.36 (1 H, d, J=8.3 Hz), 8.22 (2 H, dd, J=17.5, 8.3 Hz), 7.99-8.06 (4 H, m), 7.85-7.88 (2 H, m), 7.78 (1 H, d, J=8.8 Hz), 7.59-7.65 (3 H, m), 7.43-7.51 (3 H, m), 7.34-7.37 (1 H, m), 6.46-6.64 (4 H, m), 6.31 (1 H, d, J=13.2 Hz), 4.22 (2 H, t, J=6.3 Hz), 4.12 (2 H, t, J=6.8 Hz), 3.58 (1 H, s), 3.38 (2 H, t, J=5.8 Hz), 3.15 (2 H, t, J=5.3 Hz), 1.98 (2 H, t, J=4.9 Hz), 1.90 (6 H, s), 1.89 (6 H, s), 1.84 (2 H, q, J=7.3 Hz), 1.77 (2 H, q, J=6.8 Hz), 1.69 (2 H, quintet, J=7.3 Hz), 1.51 (2 H, quintet, J=7.3 Hz), 1.34 (2 H, quintet, J=7.3 Hz)

### Example 6: Synthesis of ICG-C9-Alkyne

In accordance with the following synthesis scheme, ICG-C9-alkyne, which is a compound in accordance with an embodiment of the present invention, was synthesized.

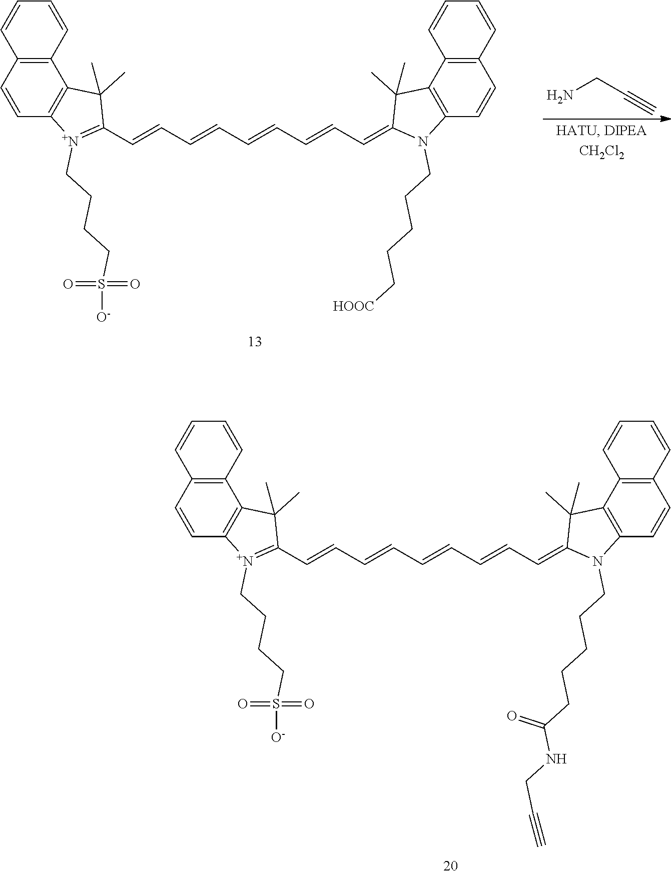

### [Compound 20]

Under an inert atmosphere, at room temperature, HATU (75 mg, 0.198 mmol), DIPEA (31 mg, 0.238 mmol), and propargylamine (9 mg, 0.159 mmol) were added to a stirred solution of the compound 13 (60 mg, 0.079 mmol) in CH₂Cl₂ (10 mL). The reaction mixture was stirred at room temperature for approximately 4 hours to 5 hours. The solvent was removed in a vacuum, and the residue was purified by column chromatography using chloroform/methanol (9:1), so that a compound 20 (43 mg, 68%) was obtained as a dark brown solid. The results of mass spectrometry (MS) and analysis of nuclear magnetic resonance (NMR) of the compound 20 are shown below.

High-Resolution Mass Spectrometry (m/s):
Calculated value: 793.3907
Measured value: 793.3790
   with respect to [M+H]⁺ of molecular formula C₅₃H₅₈N₄O₆S

¹H NMR (DMSO-d₆, 500 MHz): δ=8.68 (1 H, d, J=4.3 Hz), 8.48 (1 H, d, J=8.3 Hz), 8.19-8.24 (3 H, m), 8.00-8.06 (4 H, m), 7.88 (1 H, t, J=13.2),7.78 (1 H, d, J=8.8 Hz), 7.60-7.65 (3 H, m), 7.44-7.51 (3 H, m), 6.50-6.64 (3 H, m), 6.31 (1 H, d, J=13.6 Hz), 4.22 (2 H, t, J=6.3 Hz), 4.13 (2 H, t, J=6.8 Hz), 3.80-3.82 (2 H, m), 3.06 (1 H, s), 2.08 (2 H, t, J=7.3 Hz), 1.90 (6 H, s), 1.89 (6 H, s), 1.84 (2 H, q, J=6.8 Hz), 1.68-1.79 (4 H, m), 1.56 (2 H, quintet, J=7.3 Hz), 1.38 (2 H, quintet, J=6.8 Hz), 1.22-1.25 (2 H, m)

### Example 7: Synthesis of ICG-C11-Maleimide

In accordance with the following synthesis scheme, ICG-C11-maleimide, which is a compound in accordance with an embodiment of the present invention, was synthesized.

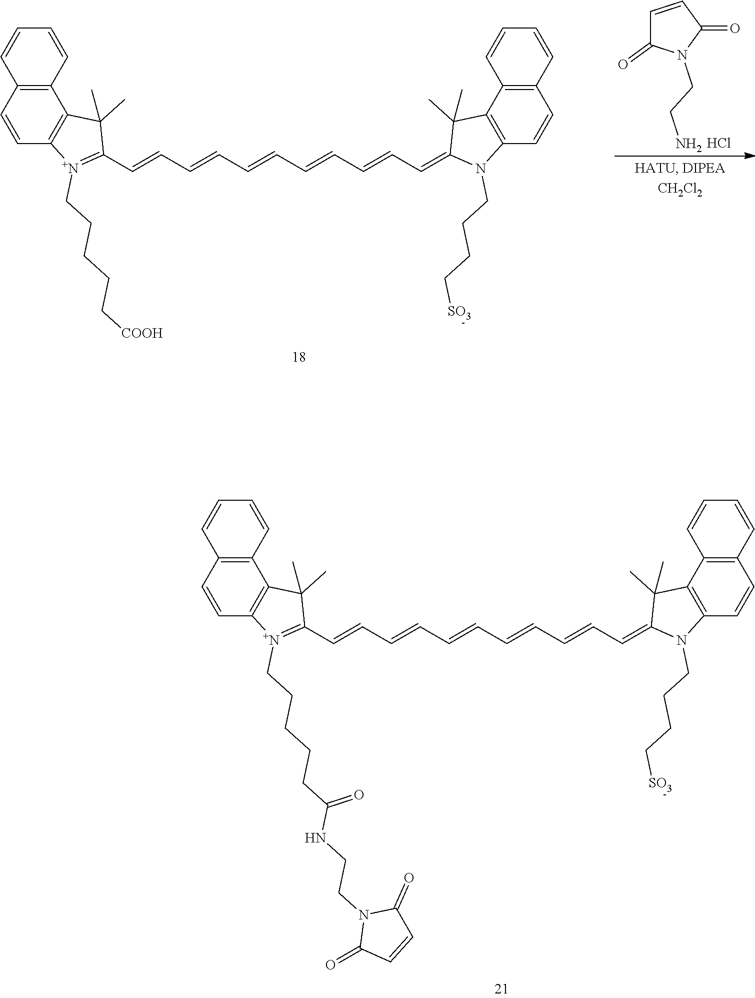

### [Compound 21]

Under an inert atmosphere, at room temperature, HATU (60 mg, 0.160 mmol), DIPEA (25 mg, 0.192 mmol), and 1-(2-aminoethyl)maleimide hydrochloride (17 mg, 0.10 mmol) were added to a stirred solution of the compound 18 (50 mg, 0.064 mmol) in dichloromethane (10 mL). The reaction mixture was stirred at room temperature overnight. The solvent was removed in a vacuum, and the residue was purified by column chromatography using chloroform/methanol (9:1), so that a compound 21 (32 mg, 55%) was obtained as a dark green solid. The results of mass spectrometry (MS) and analysis of nuclear magnetic resonance (NMR) of the compound 21 are shown below.

High-Resolution Mass Spectrometry (m/s):
Calculated value: 905.4306
Measured value: 905.4295
   with respect to [M+H]⁺ of molecular formula C₅₃H₅₈N₄O₆S

¹H NMR (DMSO-d₆, 500 MHz): δ=8.94 (2 H, s), 8.24 (1 H, d, J=8.3 Hz), 8.18 (1 H, d, J=8.8 Hz), 8.05 (2 H, t, J=8.3 Hz), 7.97-8.00 (2 H, m), 7.87-7.90 (2 H, m), 7.79 (1 H, d, J=8.8 Hz), 7.57-7.75 (5 H, m), 7.50 (1 H, t, J=7.3 Hz), 7.36-7.44 (2 H, m), 7.28 (1 H, t, J=12.7 Hz), 6.98 (2 H, s), 6.46-6.62 (4 H, m), 6.25 (1 H, d, J=13.7 Hz), 4.23 (2 H, t, J=7.3 Hz), 4.09 (2 H, t, J=5.3 Hz), 3.38 (2 H, t, J=5.8 Hz), 3.11-3.15 (2 H, m), 1.97 (2 H, t, J=7.3 Hz), 1.89 (6 H, s), 1.87 (6 H, s), 1.81-1.86 (2 H, m), 1.76 (2 H, quintet, J=6.8 Hz), 1.68 (2 H, quintet, J=6.8 Hz), 1.50 (2 H, quintet, J=7.3 Hz), 1.32-1.37 (2 H, m)

### Example 8: Synthesis of ICG-C11-Alkyne

In accordance with the following synthesis scheme, ICG-C11-alkyne, which is a compound in accordance with an embodiment of the present invention, was synthesized.

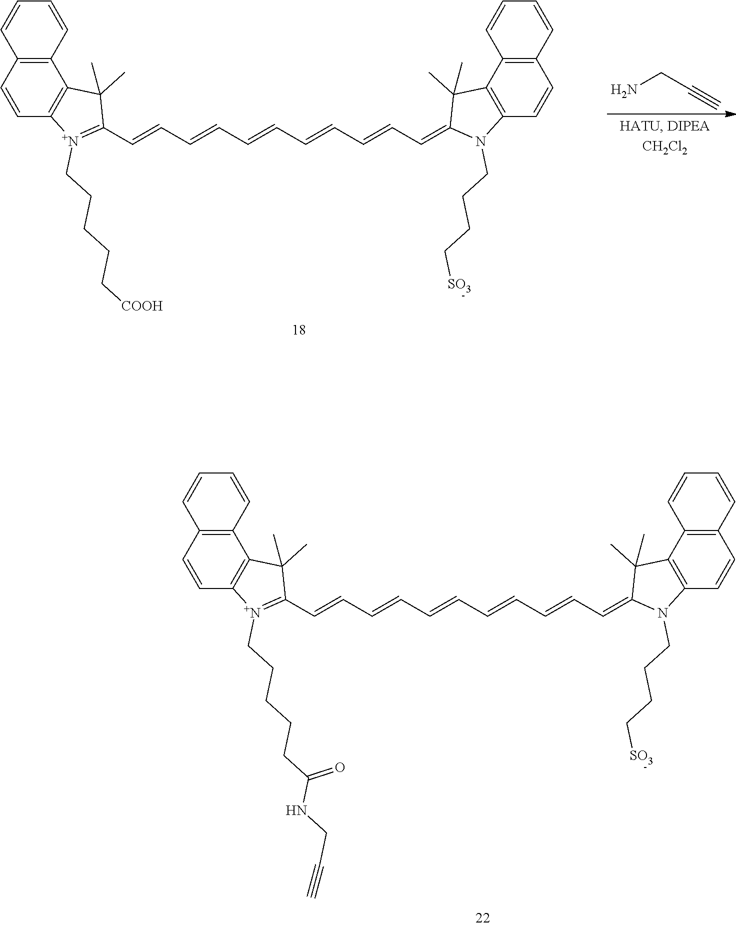

### [Compound 22]

Under an inert atmosphere, at room temperature, HATU (61 mg, 0.160 mmol), DIPEA (25 mg, 0.192 mmol), and propargylamine (7 mg, 0.128 mmol) were added to a stirred solution of the compound 18 (50 mg, 0.064 mmol) in dichloromethane (10 mL). The reaction mixture was stirred at room temperature overnight. The solvent was removed in a vacuum, and the residue was purified by column chromatography using chloroform/methanol (9:1), so that a compound 22 (30 mg, 58%) was obtained as a dark green solid. The results of mass spectrometry (MS) and analysis of nuclear magnetic resonance (NMR) of the compound 22 are shown below.

High-Resolution Mass Spectrometry (m/s):
Calculated value: 920.4142
Measured value: 920.4130
   with respect to [M+H]⁺ of molecular formula C₅₃H₅₈N₄O₆S

¹H NMR (DMSO-d₆, 500 MHz): δ=8.17-8.24 (3 H, m), 8.05 (2 H, t, J=7.8 Hz), 8.00 (2 H, d, J=8.3 Hz), 7.90 (1 H, t, J=13.2 Hz), 7.70-7.79 (2 H, m), 7.58-7.66 (3 H, m), 7.50 (1 H, t, J=7.3 Hz), 7.43 (1 H, t, J=7.3 Hz), 7.28 (1 H, t, J=13.2 Hz), 7.01 (1 H, t, J=12.7), 6.46-6.62 (4 H, m), 6.24 (1 H, d, J=13.2 Hz), 4.23 (2 H, t, J=6.8 Hz), 4.09 (2 H, t, J=5.3 Hz), 3.80-3.82 (2 H, m), 3.06 (1 H, s), 2.91 (2 H, s), 2.08 (2 H, t, J=7.3 Hz), 1.89 (6 H, s), 1.87 (6 H, s), 1.81-1.84 (2 H, m), 1.76 (2 H, quintet, J=7.3 Hz), 1.70 (2 H, quintet, J=6.8 Hz), 1.37 (2 H, quintet, J=6.8 Hz)

### Examples 9 and 10

### [Preparation of Shortwave Infrared Fluorescent Agents 1 and 2]

1 mg of the compound 4 (ICG-C9) was dissolved in 1 mL of dimethyl sulfoxide (FUJIFILM Wako Pure Chemical Corporation). Next, 0.1 mL of the solution was added to 0.9 mL of bovine serum albumin (Sigma-Aldrich Co. LLC) having a weight ratio of 1%. 1 mL of the solution subjected to dialysis (spectrum dialysis, MWCO: 50 kDa) with distilled water. Thus, an aqueous fluorescent probe solution (1 mg/mL, 1% bovine serum albumin) for imaging of blood vessels and lymph nodes was obtained. The aqueous solution was used as shortwave infrared fluorescent agent 1.

A shortwave infrared fluorescent agent 2 was prepared as in the case of the shortwave infrared fluorescent agent 1 except that the compound 10 (ICG-C11) was used instead of the compound 4.

[Imaging of Blood Vessels and Lymph Nodes with Shortwave Infrared Fluorescent Agents 1 and 2]

### <Imaging of Blood Vessel in Lower Limb and Abdomen in Mouse>

A 0.2-mL of a shortwave infrared fluorescent agent 1 was injected, through tail vein, into a hairless mouse (male, 5 weeks old; Hos: HR-1, Japan SLC, Inc.) under anesthesia with isoflurane (FUJIFILM Wako Pure Chemical Corporation). Subsequently, the blood vessels in the lower limb and the abdomen of the hairless mouse were subjected to shortwave infrared fluorescence imaging.

In addition, the blood vessels in the lower limb and the abdomen of the hairless mouse were subjected to shortwave infrared fluorescence imaging as in the shortwave infrared fluorescence imaging using the shortwave infrared fluorescent agent 1, except that the shortwave infrared fluorescent agent 2 was used instead of the shortwave infrared fluorescent agent 1. Fluorescence images were captured with use of a cooled InGaAs shortwave infrared camera (C10633-34, Peltier cooling+water cooling −70° C., dark current 132 electrons/pixel/s) manufactured by Hamamatsu Photonics K.K.

In shortwave infrared fluorescence imaging using the shortwave infrared fluorescent agent 1, the excitation wavelength was 905 nm, and the fluorescence detection wavelength was 1000 nm. In shortwave infrared fluorescence imaging using the shortwave infrared fluorescent agent 2, the excitation wavelength was 975 nm, and the fluorescence detection wavelength was 1100 nm. In each shortwave infrared fluorescence imaging, the intensity of excitation light was 20 mW/cm² to 40 mW/cm², and the exposure time was 5 seconds to 10 seconds.

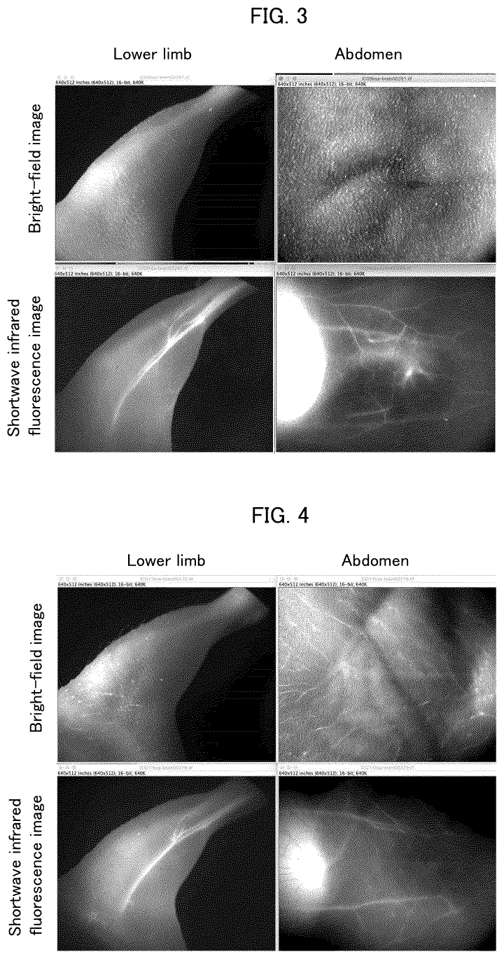

Photographs representing bright-field images and shortwave infrared fluorescence images, with the shortwave infrared fluorescent agent 1, of the lower limb and the abdomen of a hairless mouse are shown in FIG. 3. Photographs representing bright-field images and shortwave infrared fluorescence images, with the shortwave infrared fluorescent agent 2, of the lower limb and the abdomen of a hairless mouse are shown in FIG. 4. As shown in FIGS. 3 and 4, the shortwave infrared fluorescent agent 1 and the shortwave infrared fluorescent agent 2 can each form a clear image, with shortwave infrared light, of blood vessels in the limb and the abdomen. More specifically, in shortwave infrared fluorescence images with use of the shortwave infrared fluorescent agent 1 and the shortwave infrared fluorescent agent 2, the fluorescence signal intensity is high, and the amount of bleeding is small, and the images are clear. Thus, with the shortwave infrared fluorescent agent 1 and the shortwave infrared fluorescent agent 2, it is possible to achieve shortwave infrared fluorescence imaging of a living body, which is not only practical but also excellent in practicality.

### <Imaging of Cerebral Vessel of Mouse>

A 0.2-mL of a shortwave infrared fluorescent agent 1 was injected, through tail vein, into a hairless mouse (male, 5 weeks old; Hos: HR-1, Japan SLC, Inc.) under anesthesia with isoflurane (FUJIFILM Wako Pure Chemical Corporation). Subsequently, the cerebral vessels of the hairless mouse were subjected to shortwave infrared fluorescence imaging. Fluorescence images were captured using a cooled InGaAs shortwave infrared camera (C10633-34, Peltier cooling+water cooling −70° C., dark current 132 electrons/pixel/s) manufactured by Hamamatsu Photonics K.K.

In addition, the cerebral vessels of the hairless mouse were subjected to shortwave infrared fluorescence imaging as in the shortwave infrared fluorescence imaging using the shortwave infrared fluorescent agent 1, except that the shortwave infrared fluorescent agent 2 was used instead of the shortwave infrared fluorescent agent 1.

Furthermore, for comparison, the cerebral vessels of the hairless mouse were subjected to shortwave infrared fluorescence imaging as in the shortwave infrared fluorescence imaging using the shortwave infrared fluorescent agent 1, except that a shortwave infrared fluorescent agent C1 was used instead of the shortwave infrared fluorescent agent 1. The shortwave infrared fluorescent agent C1 is a fluorescent agent prepared as in the case of the shortwave infrared fluorescent agent 1, except that ICG was used instead of the compound 4.

In shortwave infrared fluorescence imaging using the shortwave infrared fluorescent agent 1, the excitation wavelength was 905 nm, and the fluorescence detection wavelength was 1000 nm. The intensity of the excitation light was 10 mW/cm², and the exposure time was 1 second.

In shortwave infrared fluorescence imaging using the shortwave infrared fluorescent agent 2, the excitation wavelength was 975 nm, and the fluorescence detection wavelength was 1100 nm. The intensity of the excitation light was 20 mW/cm² to 40 mW/cm², and the exposure time was 1 second to 2.5 seconds.

In shortwave infrared fluorescence imaging by the shortwave infrared fluorescent agent C1, the excitation wavelength was 758 nm, and the fluorescence detection wavelength was 900 nm. The intensity of the excitation light was 5 mW/cm², and the exposure time was 1 second.

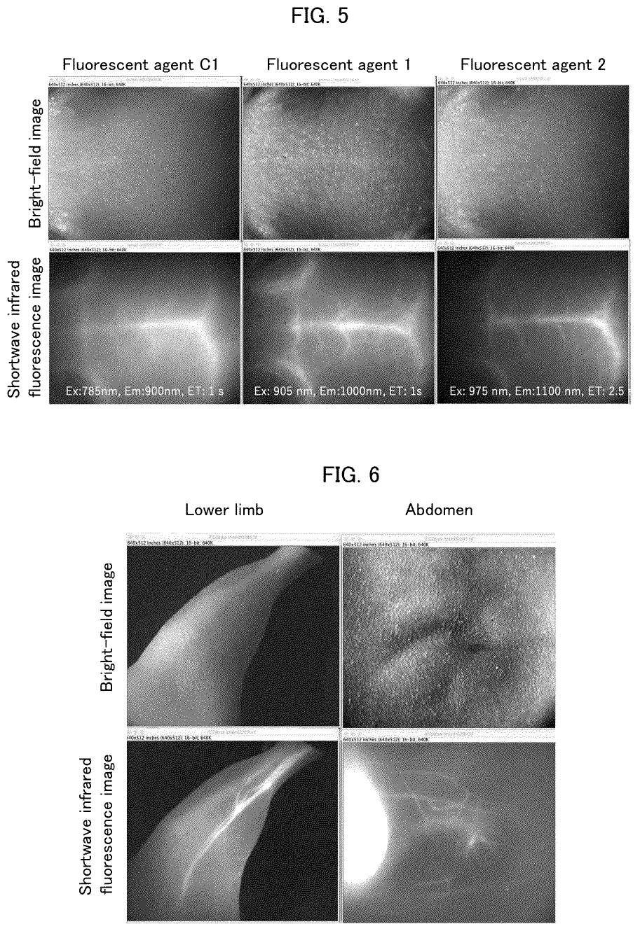

Photographs representing bright-field images and shortwave infrared fluorescence images, with the shortwave infrared fluorescent agents C1, 1, and 2, of the head top parts of hairless mice are shown in FIG. 5. As shown in FIG. 5, the shortwave infrared fluorescent agents 1 and 2, in comparison with the shortwave infrared fluorescent agent C1 containing ICG, can clearly form an image which makes it possible to observe, from the outside of shortwave infrared light, the brain vessels of the hairless mouse with the details of the cerebral vessels.

### Examples 11 and 12

### [Preparation of Shortwave Infrared Fluorescent Agents 3 and 4]

Shortwave infrared fluorescent agents 3 and 4 were each prepared. In the shortwave infrared fluorescent agent 3, the compound 14 (ICG-C9-NHS) is bonded to bovine serum albumin (BSA) via an amide group containing a carbonyl group, which is a residue of a reactive crosslinking group. In the shortwave infrared fluorescent agent 4, the compound 17 (ICG-C11-NHS) is bonded to BSA via the amide group. The preparation scheme for the shortwave infrared fluorescent agents 3 and 4 is shown below.

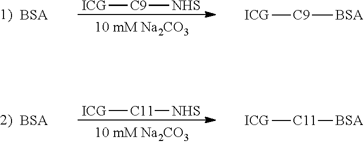

5 mg of bovine serum albumin (BSA) was dissolved in 1 mL of a 10 mM sodium carbonate solution, and the resultant product was reacted 0.1 mL of a dimethyl sulfoxide solution (1 mg/mL) of the compound 14. Thus, the compound 14 (ICG-C9-BSA) to which BSA was bonded was obtained. Purification was performed by a gel filtration column (PD10, GE Healthcare) with physiological phosphate buffer (PBS) serving as an eluate. ICG-C9-BSA was dissolved in PBS so as to achieve a concentration of 1 mg/mL. The aqueous solution obtained was used as the shortwave infrared fluorescent agent 3.

The shortwave infrared fluorescent agent 4 was prepared as in the case of the shortwave infrared fluorescent agent 3 except that the compound 17 was used instead of the compound 14.

[Imaging of Blood Vessels and Lymph Nodes with Shortwave Infrared Fluorescent Agents 3 and 4]

### <Imaging of Blood Vessel in Lower Limb and Abdomen in Mouse>

The blood vessels in the lower limb and the abdomen of the hairless mouse were subjected to shortwave infrared fluorescence imaging as in Example 5, except that the shortwave infrared fluorescent agent 3 or the shortwave infrared fluorescent agent 4 was used instead of the shortwave infrared fluorescent agent 1.

In shortwave infrared fluorescence imaging using the shortwave infrared fluorescent agent 3, the excitation wavelength was 905 nm, and the fluorescence detection wavelength was 1000 nm. The intensity of the excitation light was 10 mW/cm², and the exposure time was 5 seconds. Photographs representing bright-field images and shortwave infrared fluorescence images, with the shortwave infrared fluorescent agent 3, of the lower limb and the abdomen of a hairless mouse are shown in FIG. 6.

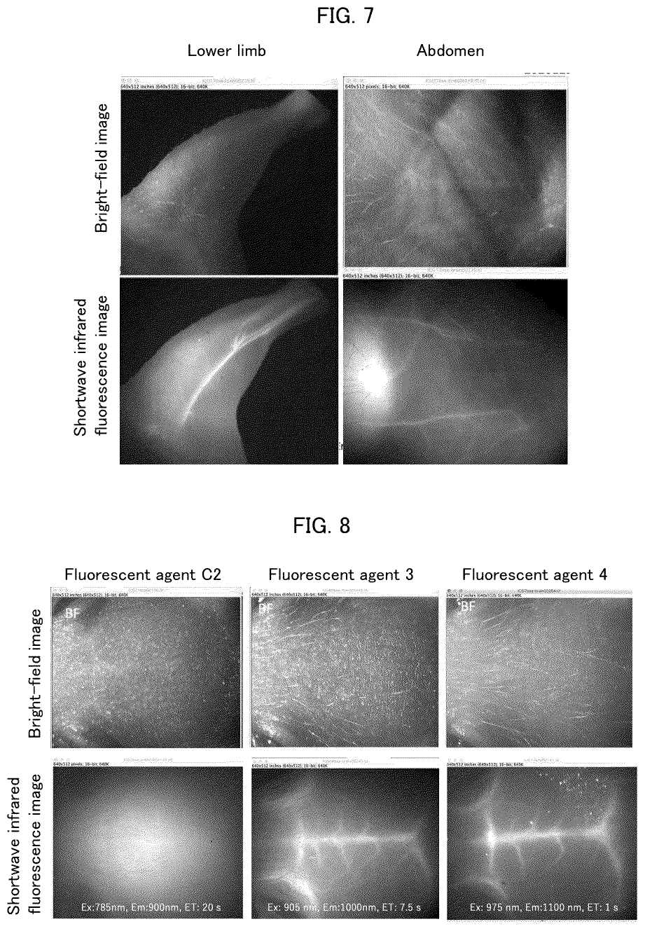

In shortwave infrared fluorescence imaging using the shortwave infrared fluorescent agent 4, the excitation wavelength was 975 nm, and the fluorescence detection wavelength was 1100 nm. The intensity of the excitation light was 20 mW/cm² to 40 mW/cm², and the exposure time was 5 seconds to 15 seconds. Photographs representing bright-field images and shortwave infrared fluorescence images, with the shortwave infrared fluorescent agent 4, of the lower limb and the abdomen of a hairless mouse are shown in FIG. 7.

As shown in FIGS. 6 and 7, the shortwave infrared fluorescent agent 3 and the shortwave infrared fluorescent agent 4 also can, as in the case of the shortwave infrared fluorescent agent 1 and the shortwave infrared fluorescent agent 2, form a clear image, with shortwave infrared light, of blood vessels in the lower limb and the abdomen.

### <Imaging of Cerebral Vessel of Mouse>

The cerebral vessels of the hairless mouse were subjected to shortwave infrared fluorescence imaging as in Example 5, except that the shortwave infrared fluorescent agent 3 or the shortwave infrared fluorescent agent 4 was used instead of the shortwave infrared fluorescent agent 1.

Furthermore, for comparison, the cerebral vessels of the hairless mouse were subjected to shortwave infrared fluorescence imaging as in Example 5, except that the shortwave infrared fluorescent agent C2 was used instead of the shortwave infrared fluorescent agent 1. The shortwave infrared fluorescent agent C2 is a fluorescent agent prepared as in the case of the shortwave infrared fluorescent agent 1, except that ICG-NHS was used instead of the compound 14. ICG-NHS can be purchased from Goryo Chemical, Inc.

In shortwave infrared fluorescence imaging using the shortwave infrared fluorescent agent 3, the excitation wavelength was 905 nm, and the fluorescence detection wavelength was 1000 nm. The intensity of the excitation light was 10 mW/cm², and the exposure time was 7.5 seconds.

In shortwave infrared fluorescence imaging using the shortwave infrared fluorescent agent 4, the excitation wavelength was 975 nm, and the fluorescence detection wavelength was 1100 nm. The intensity of the excitation light was 20 mW/cm² to 40 mW/cm², and the exposure time was 1 second.

In shortwave infrared fluorescence imaging by the shortwave infrared fluorescent agent C2, the excitation wavelength was 758 nm, and the fluorescence detection wavelength was 900 nm. The intensity of the excitation light was 5 mW/cm², and the exposure time was 20 seconds.

Photographs representing bright-field images and shortwave infrared fluorescence images, with the shortwave infrared fluorescent agents C2, 3, and 4, of the head top parts of hairless mice are shown in

FIG. 8. As shown in FIG. 8, the shortwave infrared fluorescent agents 3 and 4, as in the case of the shortwave infrared fluorescent agents 1 and 2, can also clearly form an image which makes it possible to observe, from the outside of shortwave infrared light, the brain vessels of the hairless mouse with the details of the cerebral vessels. In contrast, in an image with shortwave infrared light by the shortwave infrared fluorescent agent C2, it was difficult to externally observe the cerebral vessels of the hairless mouse.

### Examples 13 through 15

### [Preparation of Complexes 1 Through 3]

Complexes 1 and 2 were each prepared. In the complex 1, the compound 14 (ICG-C9-NHS) is bonded to an antibody (Erbitux) via an amide group containing a carbonyl group, which is a residue of a reactive crosslinking group. In the complex 2, the compound 17 (ICG-C11-NHS) is bonded to the antibody via the amide group. The preparation scheme for the complexes 1 and 2 is shown below.

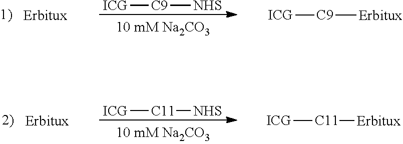

2 mg of an anti-human EGFR monoclonal antibody (Erbitux; Merck Serono) was dissolved in 1 mL of 10 mM sodium carbonate solution. The resultant product was reacted with 50 μL of a dimethyl sulfoxide solution (1 mg/mL) of the compound 14, so that a complex 1 (ICG-C9-Erbitux) in which the compound 14 was modified with Erbitux was obtained. Purification was performed by a gel filtration column.

In addition, a complex 2 (ICG-C11-Erbitux) in which the compound 17 is modified with Erbitux was prepared as in the case of the complex 1, except that the compound 17 was used instead of the compound 14.

### [Fluorescence Characteristics]

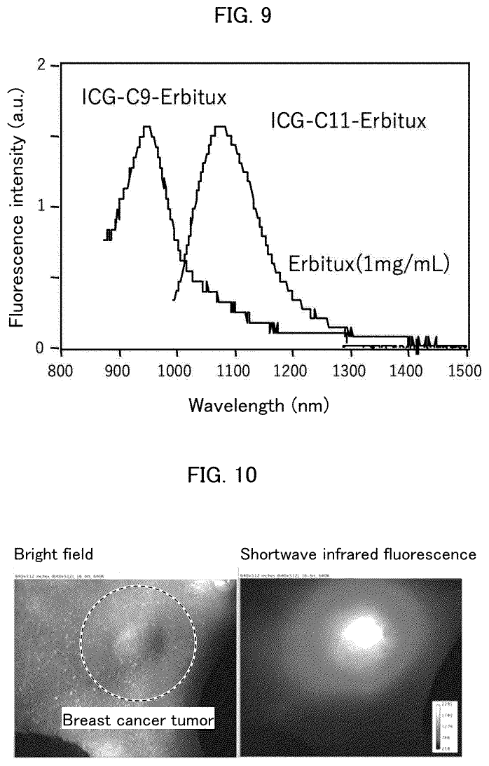

The fluorescence spectra of the aqueous solutions obtained by dissolving the complexes 1 and 2 in PBS were measured. The concentrations of the aqueous solutions measured were 1 mg/mL. The wavelengths of the excitation light were 785 nm in the complex 1 and 900 nm in the complex 2. The fluorescence spectra of the complexes 1 and 2 are shown in FIG. 9. As shown in FIG. 9, the emission peak of the fluorescence in the complex 1 was approximately 950 nm, and the emission peak of the fluorescence in the complex 2 was approximately 1100 nm.

Furthermore, a complex 3 (ICG-C11-Kadcyla), in which the compound 17 was modified with Kadcyla, was prepared as in the case of the preparation of the complex 2, except that an anti-HER2 antibody Tubulin polymerization inhibitor complex (Kadcyla (registered trademark of F. Hoffmann-La Roche, Ltd.), Chugai Pharmaceutical Co., Ltd.), which is an antibody-drug complex (ADC), was used instead of the anti-human EGFR monoclonal antibody. In the complex 3, as in the case of the complex 2, the compound 17 (ICG-C11-NHS) is bonded to the antibody in the antibody-drug complex via the amide group containing the carbonyl group. The wavelength and the fluorescence peak of the excitation light in the complex 3 are substantially identical to those in the complex 2.

### Examples 16 Through 18

### [Preparation of Shortwave Infrared Fluorescent Agents 5 Through 7]

The complex 1 was diluted with PBS, so that an aqueous solution of the complex 1 having a concentration of 1 mg/mL was obtained. The aqueous solution was used as shortwave infrared fluorescent agent 5.

A shortwave infrared fluorescent agent 6 was prepared as in the case of the shortwave infrared fluorescent agent 5, except that the complex 2 was used instead of the complex 1. A shortwave infrared fluorescent agent 7 was prepared as in the case of the shortwave infrared fluorescent agent 5, except that the complex 3 was used instead of the complex 1.

[Fluorescence Imaging of Breast Cancer Tumor with Shortwave Infrared Fluorescent Agents 5 and 6]

### <Production of Breast Cancer Model Mouse>

Human breast cancer cells (MDA-MB-468 (ATCC), the number was approximately 1.5×10⁷) were transplanted to the vicinity of the second breast of the lower limb of a 5-week-old nude mouse (BALB/c S1c-nu/n, Japan SLC, Inc.). Thus, a cancer-bearing mouse was produced as a breast cancer model mouse. The mouse was grown for 2 to 3 weeks, and the size of the tumor was grown to several millimeters.

### <Fluorescence Imaging of Breast Cancer Tumor in a Breast Cancer Model Mouse>

0.2 mL of the shortwave infrared fluorescent agent 5 was injected, through tail vein, into the cancer-bearing mouse under anesthesia with isoflurane (FUJIFILM Wako Pure Chemical Corporation). Subsequently, shortwave infrared fluorescence imaging of the breast cancer tumor of the cancer-bearing mouse was performed in vivo. Thereafter, the tumor-bearing mouse were dissected, and the breast cancer tumor and organs (heart, kidney, spleen, and liver) of the cancer-bearing mouse were taken out. Then, shortwave infrared fluorescence imaging of the breast cancer tumor and the organs thus taken out was performed ex vivo.

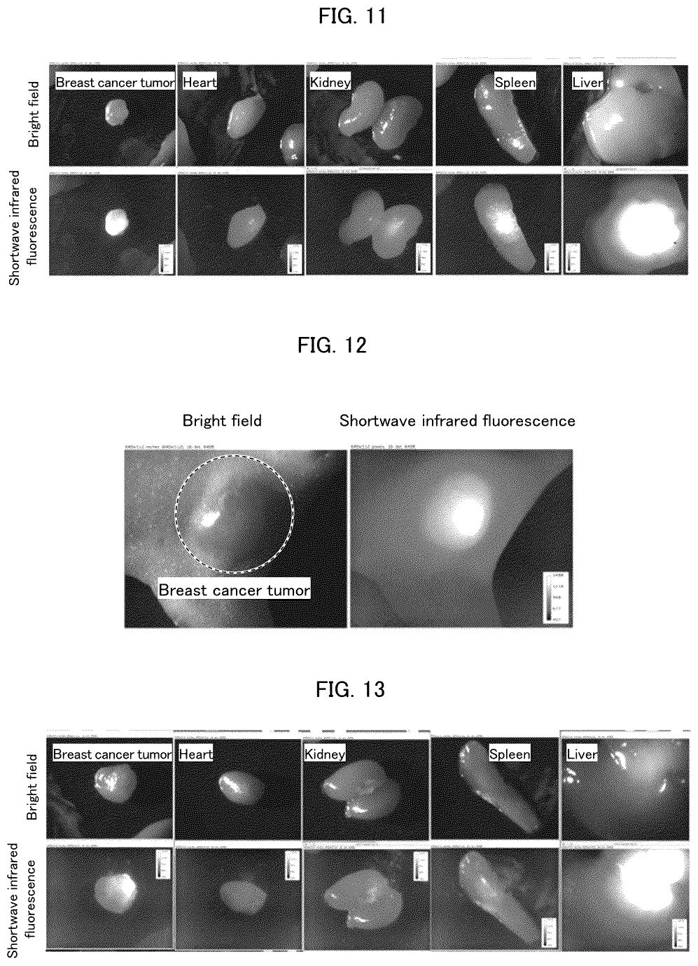

In shortwave infrared fluorescence imaging using the shortwave infrared fluorescent agent 5, the excitation wavelength was 905 nm, and the fluorescence detection wavelength was 1000 nm. The intensity of the excitation light was 10 mW/cm², and the exposure time was 15 seconds. Photographs representing an in vivo bright-field image and an in vivo shortwave infrared fluorescence image, with the shortwave infrared fluorescent agent 5, of the breast cancer tumor in the nude mouse are shown in FIG. 10. Photographs representing ex vivo bright-field images and ex vivo shortwave infrared fluorescence images, with the shortwave infrared fluorescent agent 5, of the breast cancer tumor, heart, kidney, spleen and liver of the nude mouse, are shown in FIG. 11.

The in vivo and ex vivo shortwave infrared fluorescence imaging was performed as in the case of the shortwave infrared fluorescence imaging with the shortwave infrared fluorescent agent 5, except that the shortwave infrared fluorescent agent 6 was used instead of the shortwave infrared fluorescent agent 5.

In shortwave infrared fluorescence imaging using the shortwave infrared fluorescent agent 6, the excitation wavelength was 975 nm, and the fluorescence detection wavelength was 1100 nm. The intensity of the excitation light was 20 mW/cm² to 40 mW/cm², and the exposure time was 15 seconds to 30 seconds. Photographs representing an in vivo bright-field image and an in vivo shortwave infrared fluorescence image, with the shortwave infrared fluorescent agent 6, of the breast cancer tumor in the nude mouse are shown in FIG. 12. Photographs representing ex vivo bright-field images and ex vivo shortwave infrared fluorescence images, with the shortwave infrared fluorescent agent 6, of the breast cancer tumor, heart, kidney, spleen, and liver of the nude mouse are shown in FIG. 13.

As is clear from FIGS. 10, 11, 12, and 13, the breast cancer tumor emitted shortwave infrared fluorescence due to the accumulation in the complexes 1 or complexes 2. This indicates that the shortwave infrared fluorescent agent 5 and the shortwave infrared fluorescent agent 6 were each specifically bonded to the breast cancer tumor, and that it was possible to form an image of a breast cancer tumor by the detection of shortwave infrared light.

In addition, FIGS. 10, 11, 12, and 13 clearly indicate the emission of shortwave infrared fluorescence from each organ of the heart, the kidney, the spleen, and the liver, due to the complex 1 and the complex 2. The emission from the heart and the kidney is weaker in comparison with the emission from the spleen and the liver. The emission from the heart is weak presumably because blood does not stay in the heart but merely passes through the heart. The emission from the kidney is weak presumably because the complexes that reached the kidney is excreted to the outside of the body as urine, and the amount of the complexes stored is less than those in the spleen and the liver.

[Fluorescence Imaging of Breast Cancer Tumor with Shortwave Infrared Fluorescent Agent 7]

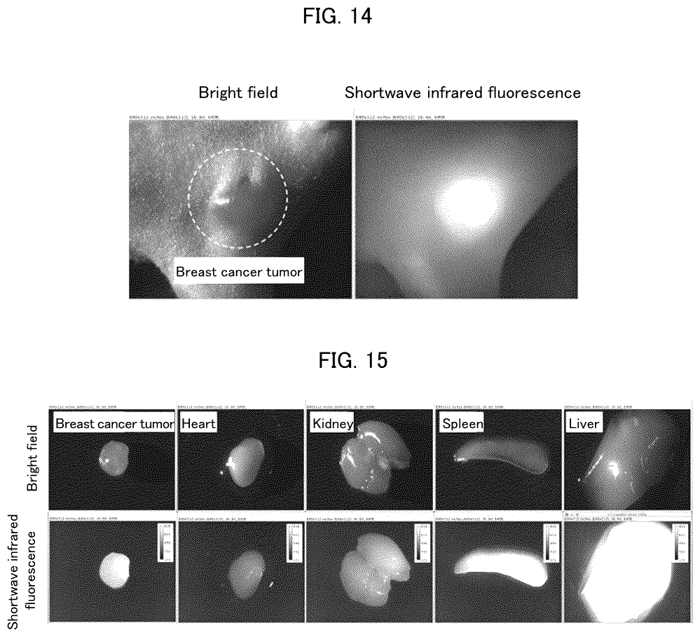

A cancer-bearing mouse was produced as in the case of the production of the above-described breast cancer model mouse, except that KPL-4 cells (provided by Kawasaki Medical School; the number was approximately 1.5×10⁷) were used as human breast cancer cells. Then, the tumor was grown to the size of several millimeters. Then, in vivo and ex vivo fluorescence imaging of the breast cancer tumor in the breast cancer model mouse was performed as in the case of the shortwave infrared fluorescent agent 6, except that the shortwave infrared fluorescent agent 7 was used instead of the shortwave infrared fluorescent agent 6. Photographs representing an in vivo bright-field image and an in vivo shortwave infrared fluorescence image, with the shortwave infrared fluorescent agent 7, of the breast cancer tumor in the nude mouse are shown in FIG. 14. Photographs representing ex vivo bright-field images and ex vivo shortwave infrared fluorescence images, with the shortwave infrared fluorescent agent 7, of the breast cancer tumor, heart, kidney, spleen, and liver of the nude mouse are shown in FIG. 15.

As can be clearly seen from FIGS. 14 and 15, the shortwave infrared fluorescent agent 7, as in the case of the shortwave infrared fluorescent agent 6, was specifically bonded to the breast cancer tumor, and it was possible to form an image of a breast cancer tumor by the detection of shortwave infrared light.

### Examples 19 and 20

### [Preparation of Complexes 4 and 5]

Complexes 4 and 5 were each prepared. In the complex 4, the compound 19 (ICG-C9-maleimide) is bonded to the antibody (Herceptin) via the succinimide group which is a residue of a reactive crosslinking group. In the complex 5, the compound 21 (ICG-C11-maleimide) is bonded to the antibody via the succinimide group. The preparation scheme for the complexes 4 and 5 is shown below.

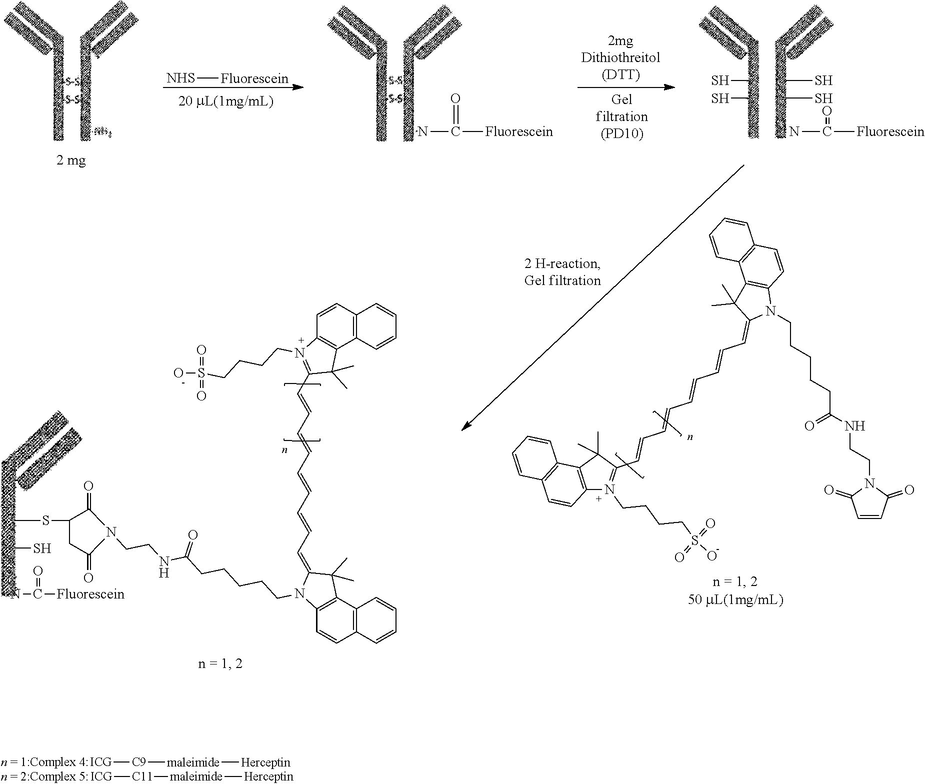

2 mg of an anti-human HER2 monoclonal antibody (Herceptin; Chugai Pharmaceutical Co., Ltd.) was dissolved in 1 mL of 10 mM sodium carbonate solution. To this solution, 20 μL of NHS-Fluorescein (Thermo Scientific) dimethyl sulfoxide solution (1 mg/mL) was added. Then, the resultant product was reacted at room temperature for 1 hour. Then, 2 mg of dithiothreitol (Tokyo Chemical Industry Co., Ltd.) was added, and the resultant product was left to stand for 30 minutes. Then, excess dithiothreitol was removed by gel filtration, with PBS serving as an eluate. To this solution, 50 μL of a dimethyl sulfoxide solution (1 mg/mL) of the compound 19 (ICG-C 9-maleimide) or the compound 21 (ICG-C 11-maleimide) was added, and the resultant product was reacted at room temperature for 2 hours. Purification was performed by a gel filtration column, so that the complex 4 (ICG-C9-maleimide-Herceptin) and the complex 5 (ICG-C11-maleimide-Herceptin) were obtained.

### Examples 21 and 22

### [Preparation of Complexes 6 and 7]

Complexes 6 and 7 were each prepared. In the complex 6, the compound 20 (ICG-C9-alkyne) is bonded to the antibody (Herceptin) via a triazole group which is a residue of the reactive crosslinking group. In the complex 7, the compound 22 (ICG-C11-alkyne) is bonded to the antibody via the triazole group. The preparation scheme for the complexes 6 and 7 is shown below.

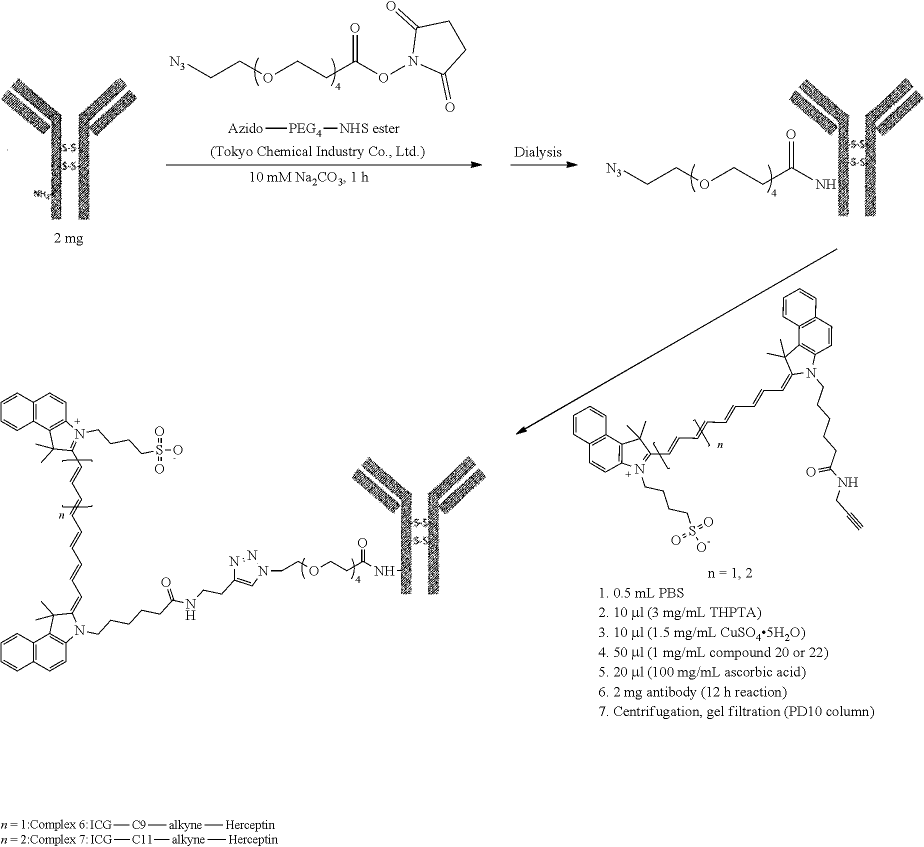

2 mg of an anti-human HER2 monoclonal antibody (Herceptin; Chugai Pharmaceutical Co., Ltd.) was dissolved in 1 mL of 10 mM sodium carbonate solution. To this solution, 20 μL of Azido-PEG4-NHSester (Tokyo Chemical Industry Co., Ltd.) dimethyl sulfoxide solution (1 mg/mL) was added. Then, the resultant product was reacted at room temperature for 1 hour. The unreacted Azido-PEG4-NHSester was removed by gel-filtration with PBS serving as an eluate, so that anti-human EGFR monoclonal antibody with modified Azido-PEG4 was prepared. Next, 10 μL of tris(3-hydroxypropyl triazolyl methyl)amine (3 mg/mL aqueous solution; Sigma-Aldrich), 10 μL of copper sulfate (0.5 mg/mL, CuSO₄·5H₂O; Nacalai Tesque, Inc.), and 50 μL of dimethyl sulfoxide solution (1 mg/mL) of the compound 20 (ICG-C 9-alkyne) or the compound 22 (ICG-C 11-alkyne) were added to a 0.5 mL solution of PBS. Furthermore, 10 μL of sodium ascorbate (100 mg/mL, Nacalai Tesque, Inc.) was added, and the resultant product was left to stand for 1 minute. To this solution, 1 mL of Azido-PEG4 modified anti-human EGFR monoclonal antibody (2 mg/mL) was added, and the resultant product was reacted at room temperature for 12 hours. Purification was performed by centrifugation and gel-filtration (PD10 columns; GE Healthcare), so that the complex 6 (ICG-C9-alkyne-Herceptin) and the complex 7 (ICG-C11-alkyne-Herceptin) were obtained.

### Examples 23 and 24

### [Preparation of Shortwave Infrared Fluorescent Agents 8 and 9]

The complex 4 was diluted with PBS, so that an aqueous solution of the complex 4 having a concentration of 1 mg/mL was obtained. The aqueous solution was used as shortwave infrared fluorescent agent 8. The complex 5 was diluted with PBS, so that an aqueous solution of the complex 5 having a concentration of 1 mg/mL was obtained. The aqueous solution was used as shortwave infrared fluorescent agent 9.

[Breast Cancer Imaging with Shortwave Infrared Fluorescent Agents 8 (Complex 4) and 9 (Complex 5)]

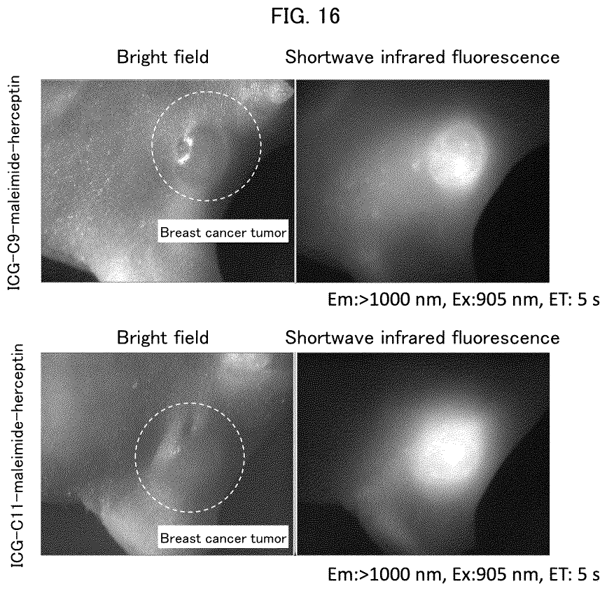

0.2 mL of the shortwave infrared fluorescent agent 8 (complex 4 having a concentration of 1 mg/mL) and 0.2 mL of the shortwave infrared fluorescent agent 9 (complex 5 having a concentration of 1 mg/mL) were injected, through tail vein, into the nude mouse to which HER2 overexpression breast cancer cells (KPL-4) were transplanted. After three days, in vivo shortwave infrared fluorescence imaging of the breast cancer tumor of the cancer-bearing mouse was performed. The fluorescence images were captured at an excitation wavelength of 905 nm and a fluorescence detection wavelength of 1000 nm or more. The intensity of the excitation light was 20 mW/cm², and the exposure time was 5 seconds. Photographs representing in vivo bright-field images and in vivo shortwave infrared fluorescence images, with the shortwave infrared fluorescent agent 8 and the shortwave infrared fluorescent agent 9, of the breast cancer tumors in the nude mice are shown in FIG. 16. These results revealed that breast cancer tumors can be clearly detected also by the shortwave infrared fluorescent agents 8 and 9.

### Examples 25 and 26

### [Preparation of Shortwave Infrared Fluorescent Agents 10 and 11]

The complex 6 was diluted with PBS, so that an aqueous solution of the complex 6 having a concentration of 1 mg/mL was obtained. The aqueous solution was used as shortwave infrared fluorescent agent 10. The complex 7 was diluted with PBS, so that an aqueous solution of the complex 7 having a concentration of 1 mg/mL was obtained. The aqueous solution was used as shortwave infrared fluorescent agent 11.

[Breast Cancer Imaging with Shortwave Infrared Fluorescent Agents 10 (Complex 6) and 11 (Complex 7)]

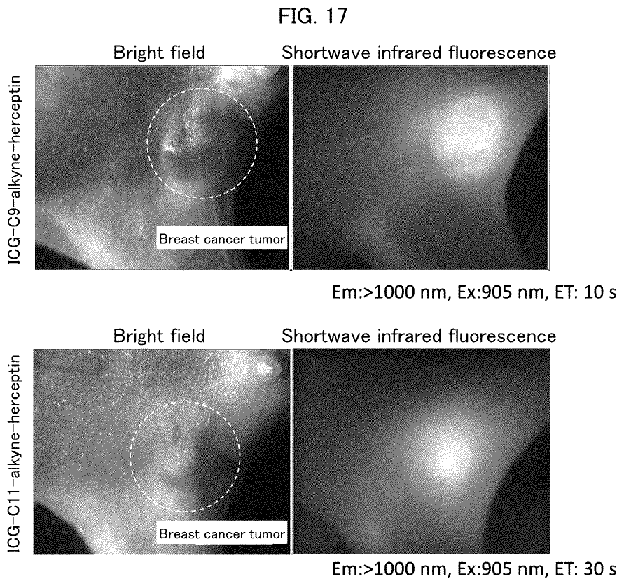

0.2 mL of the shortwave infrared fluorescent agent 10 (complex 6 having a concentration of 1 mg/mL) and 0.2 mL of the shortwave infrared fluorescent agent 11 (complex 7 having a concentration of 1 mg/mL) were injected, through tail vein, into the nude mouse to which HER2 overexpression breast cancer cells (KPL-4) were transplanted. After three days, in vivo shortwave infrared fluorescence imaging of the breast cancer tumor of the cancer-bearing mouse was performed. The fluorescence images were captured at an excitation wavelength of 905 nm and a fluorescence detection wavelength of 1000 nm or more. The intensity of the excitation light was 20 mW/cm², and the exposure times were 10 seconds (shortwave infrared fluorescent agent 10) and 30 seconds (shortwave infrared fluorescent agent 11). Photographs representing in vivo bright-field images and in vivo shortwave infrared fluorescence images, with the shortwave infrared fluorescent agent 10 and the shortwave infrared fluorescent agent 11, of the breast cancer tumors in the nude mice are shown in FIG. 17. These results revealed that breast cancer tumors can be clearly detected also by the shortwave infrared fluorescent agents 10 and 11.

### Examples 27 and 28

### [Preparation of Complexes 8 and 9]

Annexin V, which is a recombinant protein, was synthesized in accordance with the method published in the inventor's article (Setsuko Tsuboi and Takashi Jin, ChemBioChem 18, 2231-2235, 2017). To a PBS-solution (1 mg/mL) of 1 mL of Annexin V, 50 μL of dimethyl sulfoxide solution (1 mg/mL) of the compound 14 (ICG-C9-NHS) or the compound 17 (ICG-C11-NHS) was added, and the resultant product was reacted for 1 hour. Purification was performed by a gel filtration column, so that a complex 8 (ICG-C9-Annexin V) and a complex 9 (ICG-C11-Annexin V) were obtained.

### Examples 29 and 30

### [Preparation of Shortwave Infrared Fluorescent Agents 12 and 13]

The complex 8 was diluted with PBS, so that an aqueous solution of the complex 8 having a concentration of 0.5 mg/mL was obtained. The aqueous solution was used as shortwave infrared fluorescent agent 12. The complex 9 was diluted with PBS, so that an aqueous solution of the complex 9 having a concentration of 0.5 mg/mL was obtained. The aqueous solution was used as shortwave infrared fluorescent agent 13.

[Apoptosis (Cell Death) Imaging of Breast Cancer with Shortwave Infrared Fluorescent Agents 12 (Complex 8) and 13 (Complex 9)]

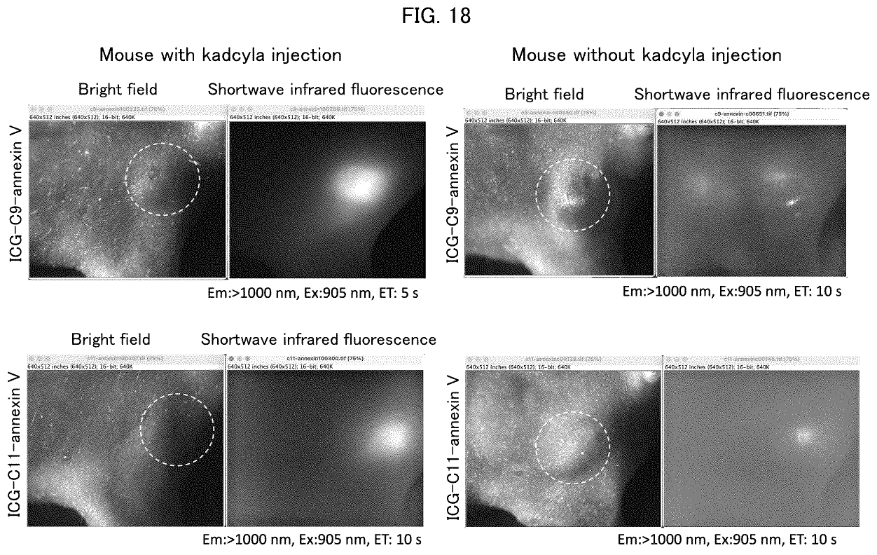

0.1 mL of Kadcyla (2 mg/mL) was injected, through tail vein, into the nude mouse to which HER2 overexpression breast cancer cells (KPL-4) were transplanted. After three days, the shortwave infrared fluorescent agent 12 (complex 8 having a concentration of 0.5 mg/mL) or the shortwave infrared fluorescent agent 13 (complex 9 having a concentration of 0.5 mg/mL) was injected into the tumor part. The next day, shortwave infrared fluorescence imaging of the breast cancer tumor of the cancer-bearing mouse was performed in vivo. The fluorescence images were captured at an excitation wavelength of 905 nm and a fluorescence detection wavelength of 1000 nm or more. The intensity of the excitation light was 20 mW/cm², and the exposure times were 5 seconds (shortwave infrared fluorescent agent 12) and 10 seconds (shortwave infrared fluorescent agent 13). Photographs representing in vivo bright-field images and in vivo shortwave infrared fluorescence images, with the shortwave infrared fluorescent agent 12 and the shortwave infrared fluorescent agent 13, of the breast cancer tumors in the nude mouse into which Kadcyla was injected (left) and the nude mouse into which Kadcyla was not injected (right) are shown in FIG. 18.

The fluorescence image revealed that apoptosis (cell death) of the tumor cells was induced by Kadcyla. The images of the control (right side) are of the nude mouse with the breast cancer tumor into which Kadcyla was not injected, and no remarkable accumulation of the shortwave infrared fluorescent agent 12 or 13 was observed.

[Over-Time Apoptosis (Cell Death) Imaging of Breast Cancer with Shortwave Infrared Fluorescent Agent 13 (Complex 9)]

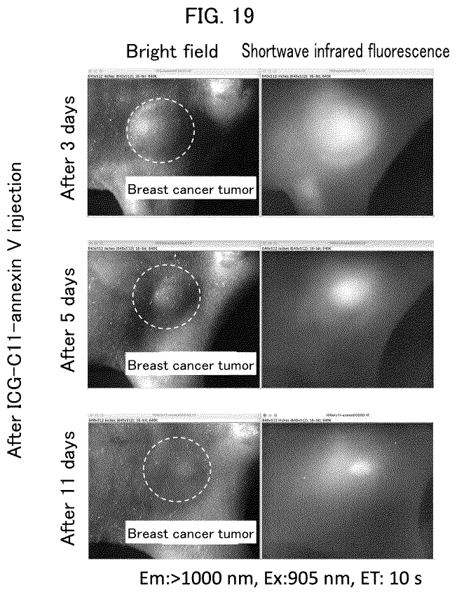

0.1 mL of Kadcyla (2 mg/mL) was injected, through tail vein, into the nude mouse to which HER2 overexpression breast cancer cells (KPL-4) were transplanted. After three days, the shortwave infrared fluorescent agent 13 (complex 9 having a concentration of 0.5 mg/mL) was injected into the tumor part. After 3 days, 5 days, and 11 days, shortwave infrared fluorescence imaging of the breast cancer tumor of the cancer-bearing mouse was performed in vivo. The fluorescence images were captured at an excitation wavelength of 905 nm and a fluorescence detection wavelength of 1000 nm or more. The intensity of the excitation light was 20 mW/cm², and the exposure time was 10 seconds. Photographs representing in vivo bright-field images and in vivo shortwave infrared fluorescence images, after specific periods of time elapsed, of the breast cancer tumor in the nude mouse with the shortwave infrared fluorescent agent 13, into which Kadcyla was injected, are shown in FIG. 19.

The fluorescence images confirmed that Kadcyla treatment induced apoptosis (cell death) of tumor cells and caused the size of the tumor to be small. These results indicate that the present shortwave infrared fluorescent agent is effective in confirming the effect of an antibody-drug complex (ADC) at an animal experimental level.

### Industrial Applicability

The present invention can be used in a shortwave infrared fluorescence imaging technique. With the present invention, the visualization of a deep portion of a living body with a resolution which greatly surpasses that of the conventional technique can be expected.

1. A compound represented by the following formula (1): 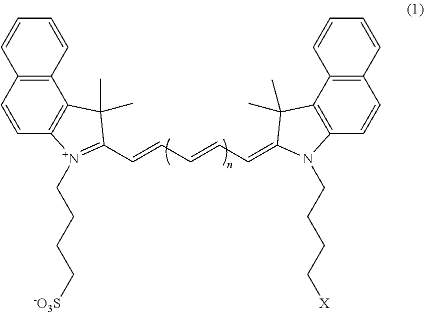
where n represents an integer of 3 to 5, X represents a salt of a sulfonic acid group or a reactive crosslinking group with respect to a molecular recognition agent.

2. The compound according to claim 1, wherein the reactive crosslinking group contains at least one organic group selected from the group consisting of an N-hydroxysuccinimide ester group, an alkynyl group, a maleimide group, and an azide group.

3. The compound according to claim 1, wherein the n is 3 or 4.

4. The compound according to claim 1, represented by one of the following formulas (2) to (9):

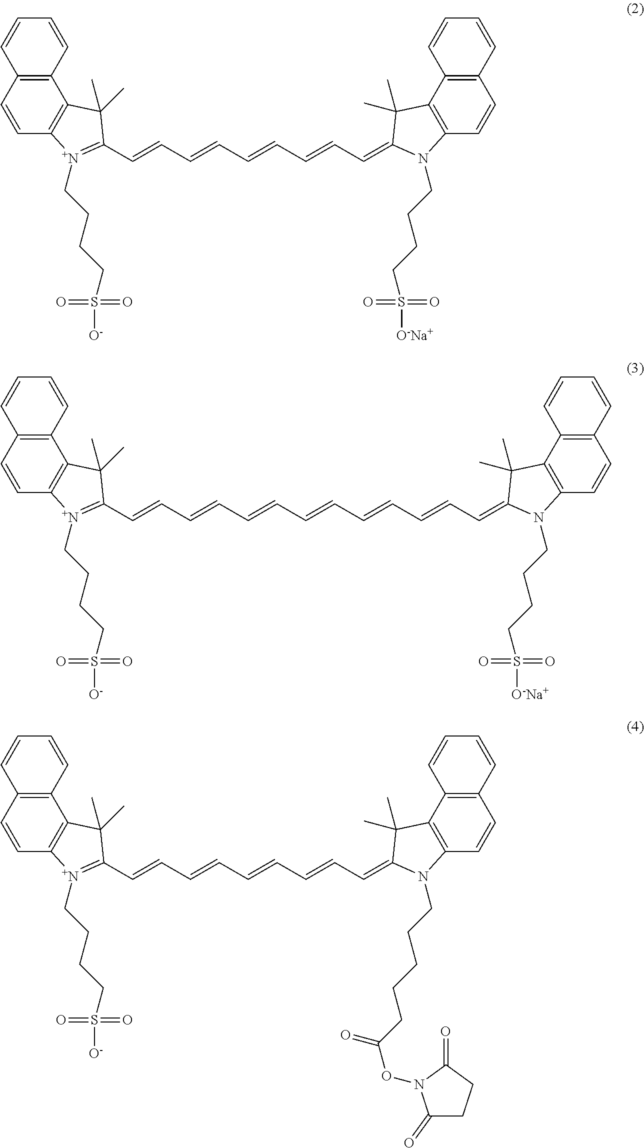

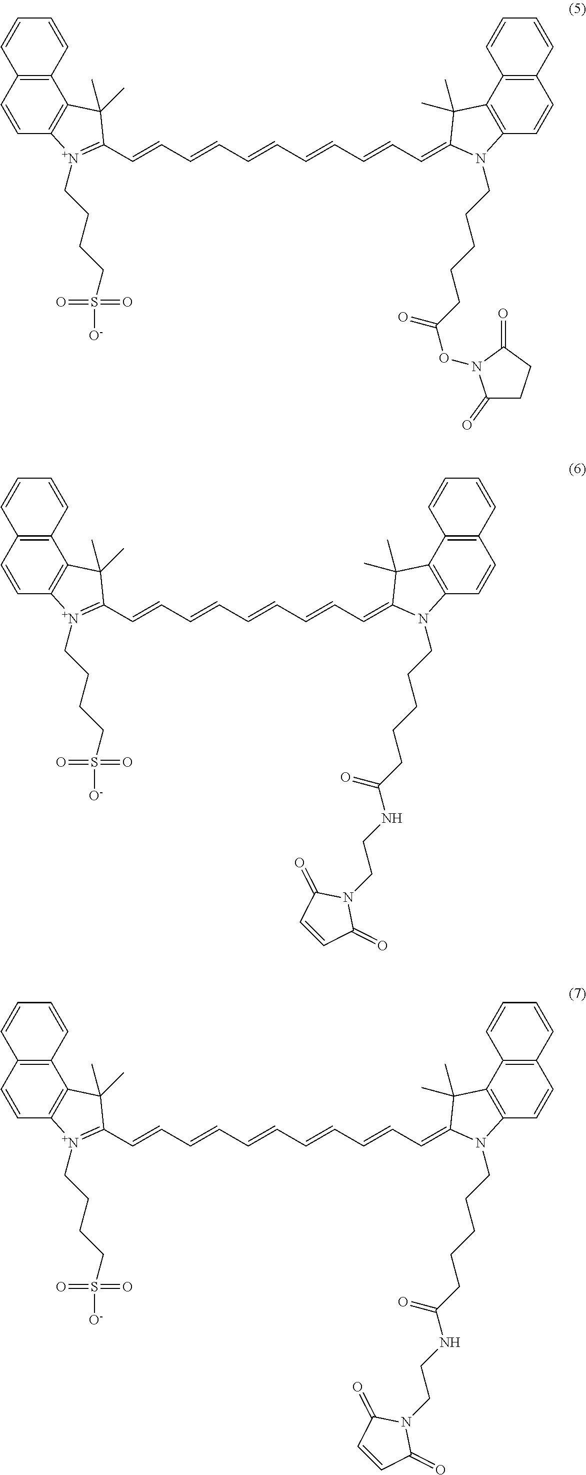 [SMILES: CC1(C)C(/C=C/C=C/C=C/C=C/C=C/C=C2/N(CCCCCC(=O)ON3C(=O)CCC3=O)c3ccc4ccccc4c3C2(C)C)=[N+](CCCCS(=O)(=O)[O-])c2ccc3ccccc3c21 ; CC1(C)C(/C=C/C=C/C=C/C=C/C=C2/N(CCCCCC(=O)NCCN3C(=O)C=CC3=O)c3ccc4ccccc4c3C2(C)C)=[N+](CCCCS(=O)(=O)[O-])c2ccc3ccccc3c21 ; CC1(C)C(/C=C/C=C/C=C/C=C/C=C/C=C2/N(CCCCCC(=O)NCCN3C(=O)C=CC3=O)c3ccc4ccccc4c3C2(C)C)=[N+](CCCCS(=O)(=O)[O-])c2ccc3ccccc3c21]

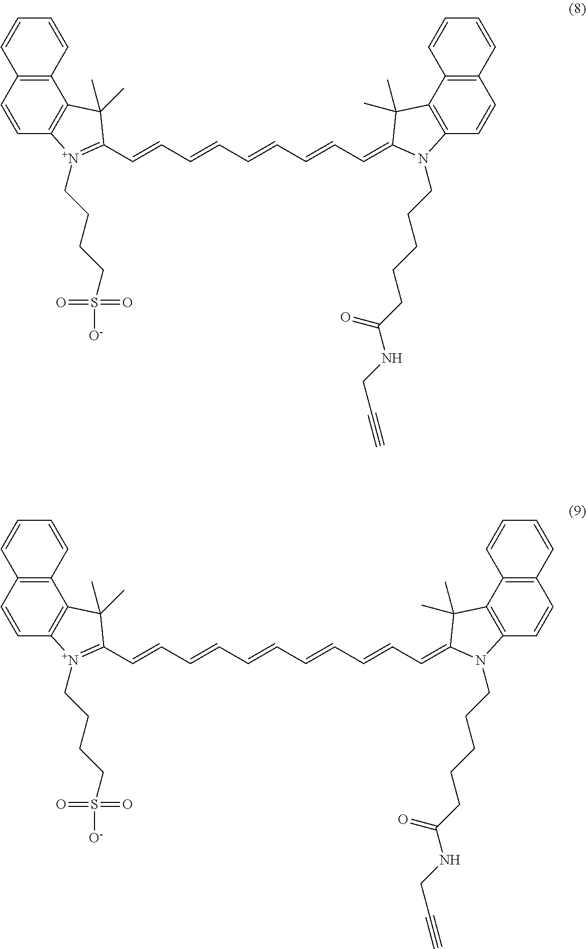 [SMILES: C#CCNC(=O)CCCCCN1/C(=C/C=C/C=C/C=C/C=C/C2=[N+](CCCCS(=O)(=O)[O-])c3ccc4ccccc4c3C2(C)C)C(C)(C)c2c1ccc1ccccc21 ; C#CCNC(=O)CCCCCN1/C(=C/C=C/C=C/C=C/C=C/C=C/C2=[N+](CCCCS(=O)(=O)[O-])c3ccc4ccccc4c3C2(C)C)C(C)(C)c2c1ccc1ccccc21]

5. A complex in which the compound according to claim 1 is bonded to a molecular recognition agent via a residue of the reactive crosslinking group in the compound.

6. The complex according to claim 5, wherein the molecular recognition agent is an antibody or a fragment of the antibody with an antigen-binding capacity.

7. A shortwave infrared fluorescent agent containing the compound according to claim 1 and/or the complex according to claim 5.

8. A method for producing a compound represented by the following formula (1), said method comprising
a first step of synthesizing a first compound represented by the following formula (1a),
a second step of replacing an anilino group in the formula (1a) with a structure represented by the following formula (1b), and
a third step of replacing a phenylimino group in the formula (1a) with a structure represented by the following formula (1c): 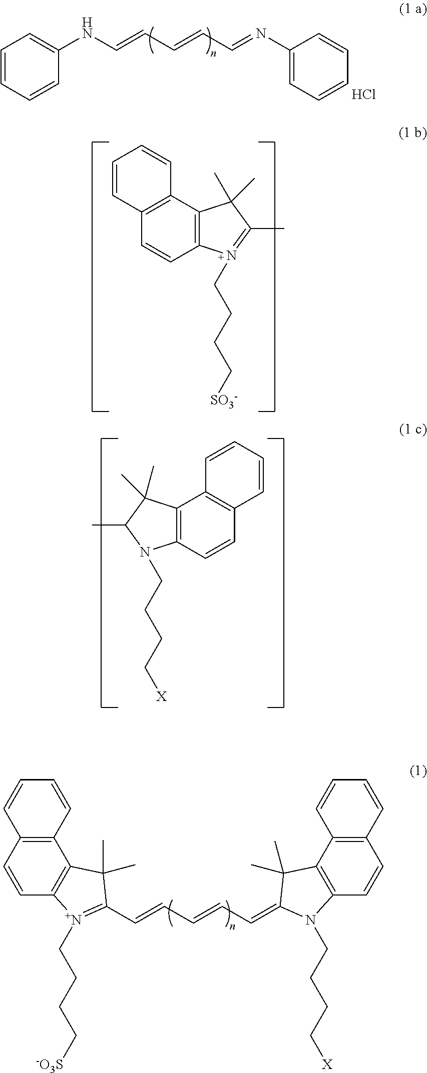
where n represents an integer of 3 to 5, X represents a salt of a sulfonic acid group or a reactive crosslinking group with respect to a molecular recognition agent.

9. The method according to claim 8, wherein:
the n is 3, and the X is a salt of a sulfonic acid group; and
the second step and the third step are carried out simultaneously.

10. The method according to claim 8, wherein:
the n is 4, and the X is a salt of a sulfonic acid group; and
the third step is carried out after the second step.

11. The method according to claim 8, wherein:
the n is 3, and the X is the reactive crosslinking group; and
the third step is carried out after the second step.

12. The method according to claim 8, wherein:
the n is 4, and the X is the reactive crosslinking group; and
the third step is carried out before the second step.
